SEQUENCE LISTING (28 sequences)

SEQ ID NO: 1 - DNA, 2322 bp, Artificial
  AN1 PolA Reconstruct (no inteins)
atgatcctcgatgctgactacatcaccgaagacggaaagcccgtcataagggtcttcaagaaggaaaagggcgagtttaagatagactacgacagggactttgagccctacatctacgccctcctgaaggacgattccgccattgaggacatcaagaagatcaccgccgagaggcacggcaccaccgttagagttacccgggcggagagggtgaagaagaagttcctcggcaggccggtggaggtctggaagctctacttcacccacccccaggacgttcccgcgatcagggacaaaatcagggagcatccggcggttgttgacatctacgagtacgacatacccttcgcgaagcgctacctcatagacaggggcttaatccctatggagggggacgaggagctcaggatgctcgccttcgacatcgagacgctctaccatgagggggaggagtttggcgaggggcctatcctgatgataagctacgccgatgaagagggggcgcgcgttatcacctggaagaatatcgacctcccctacgtggagagcgtttctactgagaaagagatgataaagcgcttcctcaaggtaatccaggagaaggatccggatgtgctcataacctacaacggcgacaacttcgactttgcttacctcaagaagcgctcagaaacgctcggcgtcaagttcatcctcggaagggacgggagcgaaccgaaaattcagcgcatgggcgaccgctttgcagtggaggtgaaggggagaatacacttcgacctctacccggttataaggaggactattaacctccccacctacaccctcgagacagtctacgaggcgattttcgggcaaccaaaggagaaggtctacgcggaagagatagcgcgggcctgggagagcggggaaggcttggaaagggtggcccgctattccatggaggacgcaaaggcaacttacgaactcggaaaagagttcttcccgatggaggcccagctctcgcgcctcgtgggccagagcctctgggatgtatcgcgctcgagcacaggaaacttagttgagtggtttctcctgaggaaggcctacgagaggaacgagctcgcgccaaacaagccggacgagagggagttagcaaggagagcggagagctacgcgggtggatatgtcaaagagcccgaaaaggggctgtgggagaacatagtctacctcgattacaaatctctctacccctcgataatcatcacccacaacgtctcccctgataccctcaacagggagggctgtagggagtacgacgtggcacctcaggtgggacaccgcttctgcaaggacttcccgggctttatcccgagcctcctcggggaccttttggaggagaggcagaaggtaaagaagaaaatgaaggccacggtggacccgatagagaggaagctcctcgactacaggcaacgcgccatcaagattctggccaacagttattacggctactacggctacgcaaatgcccgctggtactgcagggagtgcgccgagagcgttaccgcctggggcaggcagtatattgaaaccacgatgagggaaatagaggagaaatttggctttaaagtgctttacgcggataccgacggtttctttgccacgattcccggagcggacgccgaaacggtcaaaaagaaggctaaagaattcctgaactacatcaaccccagactgcccggcctgctcgagctggagtacgagggcttctacaggcgcggcttcttygtgacgaagaagaagtacgcggttatagacgaggaggacaagataacgacgcgcgggctggaaatagtaaggcgcgactggagcgagatagcgaaggagacgcaggcgagggttcttgaggcgatactcaagcacggtgacgtcgaagaggcagtaaggattgtcaaggaggtgacggaaaagctgagtaggtacgaggttccaccggagaagctcgtcatctacgagcagataacccgcgacctgagggactacagggccacggggccgcacgtggccgttgcaaaacgcctcgccgcgagggggataaaaatccggcccgggacggtcataagctacatagtgctcaaaggcccgggaagggttggggacagggcgatacccttcgacgagttcgaccctgcaaagcaccgctatgatgcggaatactacatcgagaaccaggttcttccagcggtggagaggattctgagggcctttggttaccgcaaagaggacttgaggtatcagaagacgaagcaggccggactgggggcgtggctaaaaccgaagacatga

SEQ ID NO: 2 - protein, 773 aa, Artificial
  AN1 PolA Reconstruct (no inteins)
MILDADYITEDGKPVIRVFKKEKGEFKIDYDRDFEPYIYALLKDDSAIEDIKKITAERHGTTVRVTRAERVKKKFLGRPVEVWKLYFTHPQDVPAIRDKIREHPAVVDIYEYDIPFAKRYLIDRGLIPMEGDEELRMLAFDIETLYHEGEEFGEGPILMISYADEEGARVITWKNIDLPYVESVSTEKEMIKRFLKVIQEKDPDVLITYNGDNFDFAYLKKRSETLGVKFILGRDGSEPKIQRMGDRFAVEVKGRIHFDLYPVIRRTINLPTYTLETVYEAIFGQPKEKVYAEEIARAWESGEGLERVARYSMEDAKATYELGKEFFPMEAQLSRLVGQSLWDVSRSSTGNLVEWFLLRKAYERNELAPNKPDERELARRAESYAGGYVKEPEKGLWENIVYLDYKSLYPSIIITHNVSPDTLNREGCREYDVAPQVGHRFCKDFPGFIPSLLGDLLEERQKVKKKMKATVDPIERKLLDYRQRAIKILANSYYGYYGYANARWYCRECAESVTAWGRQYIETTMREIEEKFGFKVLYADTDGFFATIPGADAETVKKKAKEFLNYINPRLPGLLELEYEGFYRRGFFVTKKKYAVIDEEDKITTRGLEIVRRDWSEIAKETQARVLEAILKHGDVEEAVRIVKEVTEKLSRYEVPPEKLVIYEQITRDLRDYRATGPHVAVAKRLAARGIKIRPGTVISYIVLKGPGRVGDRAIPFDEFDPAKHRYDAEYYIENQVLPAVERILRAFGYRKEDLRYQKTKQAGLGAWLKPKT

SEQ ID NO: 3 - DNA, 29 bp, Artificial
  ARCHPOLF1
  misc_feature (21)..(21): n is a, c, g, or t
tactacggatacgccaargcnagrtggta

SEQ ID NO: 4 - DNA, 29 bp, Artificial
  ARCHPOLF2
  misc_feature (21)..(21): n is a, c, g, or t
  misc_feature (24)..(24): n is a, c, g, or t
tactacggatacgccaargcncgntggta

SEQ ID NO: 5 - DNA, 29 bp, Artificial
  ARCHPOLR
  misc_feature (18)..(18): n is a, c, g, or t
  misc_feature (21)..(21): d is a, g, or t
gcggggagaacctggttntcdatrtarta

SEQ ID NO: 6 - DNA, 26 bp, Artificial
  THERMOPOLF1
  misc_feature (18)..(18): y is t or c
  misc_feature (21)..(21): n is a, c, g, or t
  misc_feature (24)..(24): y is t or c
tggattatgatcctcgayacngayta

SEQ ID NO: 7 - DNA, 23 bp, Artificial
  THERMOPOLF2
  misc_feature (15)..(15): n is a, c, g, or t
  misc_feature (21)..(21): r is g or a
agggagttcttcccnatggargc

SEQ ID NO: 8 - DNA, 17 bp, Artificial
  AN1.R1
ggcggtaacgctctcgg

SEQ ID NO: 9 - DNA, 18 bp, Artificial
  AN1.R2
ccggtgacactatccgcg

SEQ ID NO: 10 - DNA, 22 bp, Artificial
  AN1.R4
tagagcttccagacctccaccg

SEQ ID NO: 11 - DNA, 22 bp, Artificial
  AN1.F1
gcgatacccttcgacgagttcg

SEQ ID NO: 12 - DNA, 19 bp, Artificial
  AN1.F3
agatccgagaccatgcccg

SEQ ID NO: 13 - protein, 742 aa, Thermococcus hydrothermalis
FEPYIYALLKDDSAIEEVKKITAGRHGRVVKVKRAEKVKKKFLGRPIEVWKLYFTHPQDVPAIRDEIRRHSAVVDIYEYDIPFAKRYLIDKGLIPMEGDEELKMMSFDIETLYHEGEEFGTGPILMISYADEGEARVITWKKIDLPYVEVVSTEKEMIKRFLKVVKEKDPDVLITYNGDNFDFAYLKKRCEKIGIKFTLRRDGSEPKIQRMGDRFAVEVKGRIHFDLYPVIRRTINLPTYTLEAVYEAVFGTPKEKVYPEEITTAWETGEGLERVARYSMEDAKVTYELGREFFPMEAQLSRLIGQSLWDVSRSSTGNLVEWFLLRKAYERNEIAPNKPDERELARRRGGYAGGYVKEPERGLWDNIVYLDFMSLYPSIIITHNVSPDTFNREGCKEYDTAPQVGHKFCKDVQGFIPSLLGALLDERQKIKKRMKASIDPLEKKLLDYRQKAIKILANSYYGYYGYARARWYCKECAESVTAWGRDYIETTIHEIEERFGFKVLYADTDGFFATIPGADAETVKKKAKEFLKYINAKLPGLLELEYEGFYVRGFFVTKKKYAVIDEEGKITTRGLEIVRRDWSEIAKETQARVLEAILRHGDVEEAVRIVKDVTEKLSKYEVPPEKLVIHEQITRELKDYKATGPHVAIAKRLAARGIKIRPGTVISYIVLKGSGRIGDRAIPFDEFDPTKHRYDAEYYIENQVLPAVERILKAFGYKKEELRYQKTRQVGLGAWLKLKGKK

SEQ ID NO: 14 - protein, 775 aa, Thermococcus sp. 9oN-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 15 - protein, 775 aa, Thermococcus sp. GE8
MILDTDYITEDGKPVIRVFKKENGEFKIEYDRNFEPYFYALLKDDSAIEEVKKITAKRHGTVVKVKRAEKVKKKFLGRPIEVWKLYFTHPQDVPAIRDKIREHPAVIDIYEYDIPFAKRYLIDKGLIPMEGDEKLKMLAFDIETLYHEGEEFAEGPILMISYADEEGARVITWKKVDLPYVDVVSTEKEMIKRFLRVVKEKDPDVLITYNGDNFDFAYLKRRSEKLGVKFILGRDGSEPKIQRMGDRFAVEVKGRIHFDLYPVIRRTINLPTYTLEAVYEAIFGKPKEKVYAEEIATAWETGEGLERVARYSMEDAKVTFELGKEFFPMEAQLSRLIGQSLWDVSRSSTGNLVEWFLLRKAYERNELAPNKPDERELARRRQSYAGGYVKEPERGLWNNIVYLDFRSLYPSIIITHNVSPDTLNREGCKEYDVAPQVGHKFCKDFPGFIPSLLGDLLEERQKIKRKMRATIDPVEKKLLDYRQRAIKILANSYYGYYGYAKARWYCRECAESVTAWGRSYIETTIREIEEKFGFKVLYADTDGFFATIPGADAETVKKKAMEFLKYINAKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTKQVGLGAWLKVKGKK

SEQ ID NO: 16 - protein, 774 aa, Thermococcus fumicolans
MILDTDYITEDGRPVIRVFKKENGEFKIEYDRDFEPYIYALLKDDSAIEDVKKITASRHGTTVRVVRAGKVKKKFLGRPIEVWKLYFTHPQDVPAIRDKIREHPAVVDIYEYDIPFAKRYLIDKGLIPMEGDEELKMLAFDIETLYHEGEEFAEGPILMISYADEEGARVITWKKIDLPYVDVVSTEKEMIKRFLKVVKEKDPDVLITYNGDNFDFAYLKKRSEKLGVKFILGRDGSEPKIQRMGDRFAVEVKGRIHFDLYPVIRHTINLPTYTLEAVYEAIFGQPKEKVYAEEIAQAWETGEGLERVARYSMEDAKVTYELGREFFPMEAQLSRLVGQSFWDVSRSSTGNLVEWYLLRKAYERNELAPNKPSGRELERRRGGYAGGYVKEPERGLWENIAYLDFRSLYPSIIISHNVSPDTLNREGCGEYDEAPQVGHRFCKDFPGFIPSLLGDLLDERQKVKKHMKATVDPIEKKLLDYRQRAIKILANSFYGYYGYAKARWYCKECAESVTAWGRQYIETTMREIEEKFGFKVLYADTDGFFATIPGADAETVKKKAREFLNYINPKLPGLLELEYEGFYRRGFFVTKKKYAVIDEEGKITTRGLEIVRRDWSEVAKETQARVLEAILRHGDVEEAVRIVKEVTEKLSKYEVPPEKLVIHEQITRELKDYKATGPHVAIAKRLAARGIKVRPGTVISYIVLKGSGRIGDRTIPFDEFDPTKHRYDAEYYIENQVLPAVERILKAFGYKKEDLRYQKTRQVGLGAWLKMGKK

SEQ ID NO: 17 - protein, 773 aa, Thermococcus gorgonarius
MILDTDYITEDGKPVIRIFKKENGEFTIDYDRNFEPYIYALLKDDSPIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLIIYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYTKARWYYKECAESVTGWGREYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 18 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 19 - protein, 774 aa, Thermococcus sp. TY
MILDTDYITKDGKPIIRIFKKENGEFKIELDPHFQPYIYALLKDDSAIDEIKAIKGERHGKIVRVVDAVKVKKKFLGRDVEVWKLIFEHPQDVPALRGKIREHPAVIDIYEYDIPFAKRYLIDKGLIPMEGDEELKLMAFDIETFYHEGDEFGKGEIIMISYADEEEARVITWKNIDLPYVDVVSNEREMIKRFVQIVREKDPDVLITYNGDNFDLPYLIKRAEKLGVTLLLGRDKEHPEPKIHRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYERNELAPNKPDEEEYRRRLRTTYLGGYVKEPERGLWENIAYLDFRSLYPSIIVTHNVSPDTLEREGCKNYDVAPIVGYKFCKDFPGFIPSILGELITMRQEIKKKMKATIDPIEKKMLDYRQRAVKLLANSYYGYMGYPKARWYSKECAESVTAWGRHYIEMTIKEIEEKFGFKVLYADTDGFYATIPGEKPETIKKKAKEFLKYINSKLPGLLELEYEGFYLRGFFVAKKRYAVIDEEGRITTRGLEVVRRDWSEIAKETQAKVLEAILKEDSVEKAVEIVKDVVEEIAKYQVPLEKLVIHEQITKDLSEYKAIGPHVAIAKRLAAKGIKVRPGTIISYIVLRGSGKISDRVILLSEYDPKKHKYDPDYYIENQVLPAVLRILEAFGYRKEDLKYQSSKQVGLDAWLKK

SEQ ID NO: 20 - DNA, 4404 bp, Artificial
  AN1 PolA (including inteins)
atgatcctcgatgctgactacatcaccgaagacggaaagcccgtcataagggtcttcaagaaggaaaagggcgagtttaagatagactacgacagggactttgagccctacatctacgccctcctgaaggacgattccgccattgaggacatcaagaagatcaccgccgagaggcacggcaccaccgttagagttacccgggcggagagggtgaagaagaagttcctcggcaggccggtggaggtctggaagctctacttcacccacccccaggacgttcccgcgatcagggacaaaatcagggagcatccggcggttgttgacatctacgagtacgacatacccttcgcgaagcgctacctcatagacaggggcttaatccctatggagggggacgaggagctcaggatgctcgccttcgacatcgagacgctctaccatgagggggaggagtttggcgaggggcctatcctgatgataagctacgccgatgaagagggggcgcgcgttatcacctggaagaatatcgacctcccctacgtggagagcgtttctactgagaaagagatgataaagcgcttcctcaaggtaatccaggagaaggatccggatgtgctcataacctacaacggcgacaacttcgactttgcttacctcaagaagcgctcagaaacgctcggcgtcaagttcatcctcggaagggacgggagcgaaccgaaaattcagcgcatgggcgaccgctttgcagtggaggtgaaggggagaatacacttcgacctctacccggttataaggaggactattaacctccccacctacaccctcgagacagtctacgaggcgattttcgggcaaccaaaggagaaggtctacgcggaagagatagcgcgggcctgggagagcggggaaggcttggaaagggtggcccgctattccatggaggacgcaaaggcaacttacgaactcggaaaagagttcttcccgatggaggcccagctctcgcgcctcgtgggccagagcctctgggatgtatcgcgctcgagcacaggaaacttagttgagtggtttctcctgaggaaggcctacgagaggaacgagctcgcgccaaacaagccggacgagagggagttagcaaggagagcggagagctacgcgggtggatatgtcaaagagcccgaaaaggggctgtgggagaacatagtctacctcgattacaaatctctctacccctcgataatcatcacccacaacgtctcccctgataccctcaacagggagggctgtagggagtacgacgtggcacctcaggtgggacaccgcttctgcaaggacttcccgggctttatcccgagcctcctcggggaccttttggaggagaggcagaaggtaaagaagaaaatgaaggccacggtggacccgatagagaggaagctcctcgactacaggcaacgcgccatcaagattctggccaacagtattctgccggatgagtggatcccgctactcattaatggaaggctgaaactggtcagaatcggcgactttgtggatagtgcgatgaaagaactgaagcccatgaaaagggatgaaacggaagtccttgaagtttctggaataggtgcgatttccttcaacaggaaaaccaagagatccgagaccatgcccgtcagggccctcctgcggcaccgctacagtggaaaagtgtacgggataaagctgtcctcggggaggaagatcaaagtcaccgcgggacacagcctcttcactttcagagacggggaactcgtggagattaagggggaggaaataaaacccggcgatttcatagcggttccaggaagaattaacctcccagaaaggcaggagaggataaacctcgtggaggttctcctcggccttcctgaggaggaaaccgccgacatcgtgctgacgatcccggttaagggacgtaggaacttctttaaaggcatgctgagaacccttcgctggatttttggggaagagaaaaggcccgggacggccaggagataccttgaacacctccaaacgctcggctacgtcaggctcgggaaaatcggctacgaaatagttaacgaggaagccctgagggactacagagggctttacgagactctaaccggaaaagtgaagtacaacggcaataagagggaataccttgtgcacttcaatgacctgagggatataataagactcatgccagagaaggagcttaaggaatggaaagttgggaccctcaacggcttcaggatggagacttccattgaagtcaaggaggactttgcaaagctcctcagctattacgtcagcgagggctatgcaggaaagcagagaagccagaaaaacgggtggaactattcagttaagctttacaacaacgaccaaaacgtccttgacgacatggaaacgctcgcctcgaagttcttcggaaaggtgagacgcgggaagaattacgttgagatcccgaggaaaatggcctacgtcctctttgagagcctttgcggtactctggccgagaacaaacgggttcctgagattatattcacctcccccgagagcgtgcgctgggccttccttgagggctgctttataggggacggcgaccttcatccgggcaaaggggttagactttccacgaagagcgaggaactggtaaacggtctggtcatcttactcaactcccttggagtttccgccctcaggatatggttagacagcggggtttacagggttctcgtcaacgaagagcttccgtttttagacaagggcaagaaaaagaccccctacgtaacttcaaaggaaataccggaggaggcctttggaaaacggttccagaggaacataagcctagaaaagctccgggagaaggttgaaaagggcgagcctgatgcggaaaaggtcaagagggtcgtgtggctccttgagggagatatagtgcttgatagggttgaggaagttgcagttgatgattacgagggctacgtctacgacctgagcgttgaagagaacgagaacttcctggcaggatttggaatgctgtacgcccacaacagttattacggctactacggctacgcaaatgcccgctggtactgcagggagtgcgccgagagcgttaccgcctggggcaggcagtatattgaaaccacgatgagggaaatagaggagaaatttggctttaaagtgctttacgcggatagtgtcaccggggacaccgaggtaatcatcagaaggaacggcaggatcgagttcgttccaatcgagagactctttgagcacgttgattaccgggttggtgagaaagaatactgcgttctcagcggtgttgaagcactgacactcgacaacaggggcaggctcgtttggaagaaggttccgtacgtcatgagacataaaacggacaagagaatttaccgcgtctgggtgaccaacagccggtacctgaacgttacggaggatcactcgctaataggttatctggacggaaaatacctggagataagacccgctgatatcccaaaagatcccgacataaagctaataaccctcgcatcccccgggttgcaggaagtcgcgctcaaaactccctcaaggcttgaagagataacctatgagggctacgtctatgacattgaagttgaagggrcccacaggttctttgccaacggaatactcgttcacaacaccgacggtttctttgccacgattcccggagcggacgccgaaacggtcaaaaagaaggctaaagaattcctgaactacatcaaccccagactgcccggcctgctcgagctggagtacgagggcttctacaggcgcggcttcttygtgacgaagaagaagtacgcggttatagacgaggaggacaagataacgacgcgcgggctggaaatagtaaggcgcgactggagcgagatagcgaaggagacgcaggcgagggttcttgaggcgatactcaagcacggtgacgtcgaagaggcagtaaggattgtcaaggaggtgacggaaaagctgagtaggtacgaggttccaccggagaagctcgtcatctacgagcagataacccgcgacctgagggactacagggccacggggccgcacgtggccgttgcaaaacgcctcgccgcgagggggataaaaatccggcccgggacggtcataagctacatagtgctcaaaggcccgggaagggttggggacagggcgatacccttcgacgagttcgaccctgcaaagcaccgctatgatgcggaatactacatcgagaaccaggttcttccagcggtggagaggattctgagggcctttggttaccgcaaagaggacttgaggtatcagaagacgaagcaggccggactgggggcgtggctaaaaccgaagacatga

SEQ ID NO: 21 - DNA, 30 bp, Artificial
  AN1pETF
gggtgggtcgacatgatcctcgatgctgac

SEQ ID NO: 22 - DNA, 32 bp, Artificial
  AN1pETR
cggattgcggccgctcatgtcttcggttttag

SEQ ID NO: 23 - DNA, 34 bp, Artificial
  Extein2F
ccatcaagattctggccaacagttattacggcta

SEQ ID NO: 24 - DNA, 17 bp, Artificial
  Extein3F
accgacggtttctttgc

SEQ ID NO: 25 - DNA, 17 bp, Artificial
  Extein1RB
ggccagaatcttgatgg

SEQ ID NO: 26 - DNA, 34 bp, Artificial
  Extein2RB
gcaaagaaaccgtcggtatccgcgtaaagcactt

SEQ ID NO: 27 - DNA, 84 bp, Artificial
  KP_PALIN_81
ctcctggatcgacttcagtccgacgatgattacatcagctcctggatcgacttcactccgcacccgctaccaacaacagtaccc

SEQ ID NO: 28 - DNA, 81 bp, Artificial
  KP_PALIN_cont
ctcctggatcgacttcagtccgatgattagatgtcgtcctggatcgacttcactccgcacccgctaccaacaacagtaccc

(19) United States
(12) Patent Application Publication
(10) Pub. No.: US 2007/0009924 A1
(43) Pub. Date: Jan. 11, 2007

(54) Thermococcus zilligii DNA polymerases and variants thereof

(76) Inventors: Jun E. Lee, San Diego, CA (US); Kyusung Park, Vista, CA (US); Katherine R. Griffiths, Hornsby (AU); Moreland D. Gibbs, Turramurra (AU); Peter L. Bergquist, Auckland (NZ)

Correspondence Address: INVITROGEN CORPORATION;C/O INTELLEVATE, P.O. BOX 52050, MINNEAPOLIS, MN, 55402, US

(21) Appl. No.: 11/341,891

(22) Filed: Jan. 30, 2006

Related U.S. Application Data
(60) Provisional application No. 60/647,408, filed on Jan. 28, 2005.

(51) Int. Cl. C12Q 1/68; C07H 21/04; C12P 21/06; C12P 19/34; C12N 9/22; C12N 1/21; C12N 15/74

(52) U.S. Cl. 435/6; 435/69.1; 435/91.2; 435/199; 435/252.3; 435/471; 536/23.2

(57) ABSTRACT

Native and variant Thermococcus zilligii DNA polymerases are disclosed, as are methods for using the same for nucleic acid synthesis, DNA sequencing, nucleic acid amplification and cDNA synthesis.

### CROSS REFERENCE TO RELATED APPLICATIONS

The present invention claims the benefit of U.S. Provisional Patent Application No. 60/647,408, filed Jan. 28, 2005, the disclosure of which is incorporated herein by reference in its entirety.

### FIELD OF THE INVENTION

The present invention relates to thermostable DNA polymerases from the thermophilic archaeon Thermococcus zilligii (Tzi), and variants thereof. These polymerases may be used, e.g., for nucleic acid synthesis, sequencing and amplification.

### BACKGROUND OF THE INVENTION

DNA polymerases synthesize formation of DNA molecules that are complementary to all or a portion of a nucleic acid template. Upon hybridization of a primer to the single-stranded template, polymerases synthesize DNA in the 5′ to 3′ direction, i.e., successively adding nucleotides to the 3′-hydroxyl group of the growing strand. Thus, for example, in the presence of deoxynucleoside triphosphates (dNTPs) and a primer, a new DNA molecule, complementary to the single stranded nucleic acid template, can be synthesized. Typically an RNA or DNA template is used for synthesizing a complementary DNA molecule. However, other templates, such as chimeric templates or modified nucleic acid templates are also usable for synthesizing complementary molecules of polymerized nucleic acids. A DNA-dependent DNA polymerase utilizes a DNA template and produces a DNA molecule complementary to at least a portion of the template. An RNA-dependent DNA polymerase, i.e. a reverse transcriptase, utilizes an RNA template to produce a DNA strand complementary to at least a portion of the template, i.e., a cDNA. A common application of reverse transcriptase has been to transcribe mRNA into cDNA. Some DNA polymerases have both DNA-dependent DNA polymerase activity and RNA-dependent DNA polymerase activity.

In addition to a polymerase activity, DNA polymerases may possess one or more additional catalytic activities. Typically, DNA polymerases may have a 3′-5′ exonuclease (“proofreading”) and a 5′-3′ exonuclease activity. Each of these activities has been localized to a particular region or domain of the protein. For example, when E. coli polymerase I (pol I) is cleaved into two fragments by subtilisin, the larger (“Klenow”) fragment has 3′-5′ exonuclease and DNA polymerase activities and the smaller fragment has 5′-3′ exonuclease activity.

DNA polymerases have been isolated from a variety of mesophilic and thermophilic organisms. DNA polymerases from thermophilic organisms typically have a higher optimum temperature for polymerization activity than enzymes isolated from mesophilic organisms. Thermostable DNA polymerases have been discovered in a number of thermophilic bacterial species, including Thermus aquaticus (Taq), Thermus thermophilus (Tth), and species of the Bacillus, Thermococcus, Sulfolobus and Pyrococcus genera. In addition, thermostable DNA polymerases from a variety of other thermophiles are described in co-pending U.S. patent application Ser. No. 10/244,081, filed Sep. 16, 2002, the entire contents of which are incorporated herein by reference. Thermostable DNA polymerases have been exploited in numerous applications, including the polymerase chain reaction (PCR).

PCR is used to amplify a target nucleic acid. PCR utilizes denaturation of the target DNA, hybridization of oligonucleotide primers to specific sequences on opposite strands of the target DNA molecule, and subsequent extension of these primers with a DNA polymerase, usually a thermostable DNA polymerase, to generate two new strands of DNA which then serve as templates for a further round of hybridization and extension. If the polymerase is thermostable, then there is no need to add fresh polymerase after every denaturation step since heat will not have destroyed the polymerase activity. In RT-PCR, a DNA primer is hybridized to a strand of the target RNA molecule, and subsequent extension of this primer with a reverse transcriptase generates a new strand of DNA (i.e., cDNA), which can serve as a template for PCR.

Thermostable DNA polymerases from Thermus aquaticus (Taq) made PCR feasible. Other thermostable polymerases having different properties (e.g., higher or lower fidelity; additional, enhanced, fewer or reduced catalytic activities; altered substrate use or preference; or different cofactor requirements) suitable for particular applications have been isolated from other organisms and/or made using recombinant DNA techniques.

### SUMMARY OF THE INVENTION

The invention features novel DNA polymerases useful for nucleic acid synthesis, sequencing, and/or amplification, namely native DNA polymerases from the thermophilic bacteria Thermococcus zilligii (Tzi) and variants thereof.

In one embodiment, the present invention provides isolated native or variant Thermococcus zilligii (Tzi) DNA polymerases having an amino acid sequence at least 80% identical to SEQ ID NO: 2. In suitable embodiments, such polymerases will have a molecular weight of about 90 kDa, and be stable at 95° C. for about 60 minutes. The present invention also provides expression vectors encoding for such DNA polymerases and host cells comprising the vectors. In another embodiment, the present invention provides an isolated monoclonal antibody that binds to the Tzi DNA polymerases of the present invention.

In a further embodiment, the present invention provides methods of synthesizing a double-stranded DNA molecule, comprising: hybridizing a primer to a first DNA molecule; and incubating the DNA molecule in the presence of one or more deoxy- and/or didexoyribonucleoside triphosphates and at least one of the Tzi DNA polymerases of the present invention under conditions sufficient to synthesize a second DNA molecule complementary to all or a portion of the first DNA molecule.

In an additional embodiment, the present invention provides methods of amplifying a double stranded DNA molecule, comprising: providing a first and second primer, wherein the first primer is complementary to a sequence at or near the 3′-terminus of the first strand of the DNA molecule and the second primer is complementary to a sequence at or near the 3′-terminus of the second strand of the DNA molecule; hybridizing the first primer to the first strand and the second primer to the second strand in the presence of at least one of the DNA polymerases of the present invention, under conditions such that the third strand complementary to the first strand and a fourth strand complementary to the second strand are synthesized; denaturing the first and third strands and the second and fourth strands; and repeating these steps one or more times.

The present invention also provides methods of preparing cDNA from mRNA, comprising: contacting mRNA with an oligo(dT) primer or other complementary primer to form a hybrid; and contacting the hybrid formed with the DNA polymerase of the present invention and dATP, dCTP, dGTP and dTTP, whereby a cDNA-RNA hybrid is obtained.

In a further embodiment, the present invention provides methods of preparing dsDNA from mRNA, comprising: contacting mRNA with an oligo(dT) primer or other complementary primer to form a hybrid; and contacting the hybrid formed with at least one of the DNA polymerases of the present invention, dATP, dCTP, dGTP and dTTP, and an oligonucleotide or primer which is complementary to the first strand cDNA; whereby dsDNA is obtained.

### BRIEF DESCRIPTION OF THE DRAWINGS

FIG. 1 is a schematic diagram showing isolation of the T zilligii polymerase gene and predicted extein/intein boundaries.

FIG. 2 is a schematic diagram showing the extein/intein sizes of T. zilligii, T. sp. GE8 PolA and T. fumicolans PolA.

FIG. 3 is a phylogram showing the evolutionary relationships of Thermococcus DNA polymerases.

FIG. 4 is a schematic diagram of the T. zilligii polymerase gene, predicted extein/intein boundaries and proposed reconstruction of the gene lacking inteins.

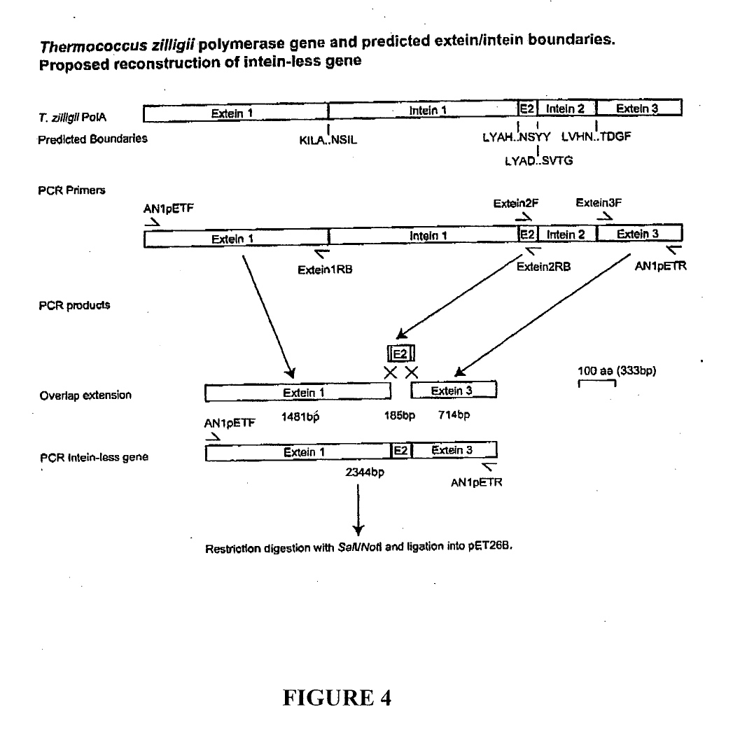

### DETAILED DESCRIPTION OF THE INVENTION

The present invention is based on the discovery of a novel, high fidelity, thermostable DNA polymerase from the thermophilic bacterium Thermococcus zilligii (Tzi) and variants (e.g., homologs and mutants) thereof. Compositions and reaction mixtures containing such novel polymerases also are described herein, as are methods for nucleic acid synthesis, sequencing and amplification using the disclosed DNA polymerases. The following glossary included terms commonly used by those skilled in the art of molecular biology.

Glossary

Cloning vector. A nucleic acid molecule, for example a plasmid, cosmid or phage DNA or other DNA molecule, that is able to replicate autonomously in a host cell. A cloning vector may have one or a small number of recognition sites (e.g., recombination sites, restriction sites, topoisomerase sites, etc.) at which such DNA sequences may be manipulated in a determinable fashion without the loss of an essential biological function of the vector, and into which a nucleic acid segment of interest may be inserted in order to bring about its replication and cloning. The cloning vector may further contain a marker suitable for use in the identification of cells transformed with the cloning vector. Markers may be, for example, antibiotic resistance such as tetracycline resistance, ampicillin resistance or kanamycin resistance genes. Any other marker sequence known to those skilled in the art may be used.

Expression vector. A vector similar to a cloning vector but which is capable of enhancing the expression of a gene that has been cloned into it, after transfection into a host. The cloned gene is usually placed under the control of (i.e. operably linked to) certain control sequences such as promoter or enhancer sequences.

Host/recombinant host. Any prokaryotic cell, eukaryotic cell or microorganism that is the recipient of a replicable expression vector, cloning vector or any heterologous nucleic acid molecule which may or may not be integrated into host genomic DNA. The nucleic acid molecule may contain, a structural gene, or portion thereof, a promoter and/or an origin of replication. The terms “host” and “recombinant host” are also meant to include those host cells which have been genetically engineered to contain the heterologous nucleic acid sequences as part of the host chromosome or genome.

Promoter. A DNA sequence to which an RNA polymerase binds such that the polymerase, in the presence of the appropriate cofactors, initiates transcription at a transcriptional start site of a nucleic acid sequence to be transcribed. Promoters may include any 5′ non-coding region that may be present between the transcriptional and translational start sites. Promoters may include cis-acting transcription control elements such as enhancers and other nucleotide sequences capable of interacting with transcription factors.

Operably linked. As used herein means that the promoter or other control sequence, such as an enhancer, is positioned to affect or control transcription of a nucleic acid sequence to which it is associated in cis.

Expression. Expression is the process by which a polypeptide is produced from a nucleic acid. It may include transcription of a gene into mRNA and the translation of such mRNA into polypeptide(s).

Substantially pure. As used herein “substantially pure” refers to a protein that is essentially free from cellular contaminants which are associated with the desired protein in nature and may impair or enhance its function. Such contaminants include, but are not limited to, phosphatases, exonucleases, endonucleases or undesirable DNA polymerases. Substantially pure polypeptides can have 25% or less, 15% or less, 10% or less, 5% or less, or 1% or less contaminating cellular components. In some cases, substantially pure DNA polymerases have no detectable protein contaminants when 200 DNA polymerase units are run on a protein gel (e.g., SDS-PAGE) and stained with Coomassie blue.

Substantially isolated. As used herein “substantially isolated” refers to a polypeptide that is essentially free from contaminating proteins which may be associated with the polypeptide in nature and/or in a recombinant host. The substantially isolated peptide can have 25% or less, 15% or less, 10% or less, 5% or less, or 1% or less contaminating proteins. In some cases, substantially isolated polypeptides represent more than 75%, 85%, 90%, 95%, 98%, or 99% of the protein in a sample. The percentage of contaminating protein and/or protein of interest in a sample may be determined using techniques well known in the art (e.g., SDS-PAGE). In some cases, the substantially pure polypeptide has no detectable protein contaminants when 0.5 μg of a sample containing the polypeptide is analyzed by SDS-PAGE.

Substantially reduced. A recombinant enzyme “substantially reduced” in an enzymatic activity means that the enzyme has less than about 30%, less than about 20%, less than about 15%, less than about 10%, less than about 7.5%, less than about 5%, less than about 2% or less than about 1% of the activity of the corresponding (e.g., unmodified wild type) enzyme.

Primer. As used herein “primer” refers to a single stranded oligonucleotide that is extended by covalent bonding of nucleotide monomers during polymerization or amplification of a nucleic acid molecule.

Template. The term “template” as used herein refers to a double-stranded or single-stranded DNA or RNA substrate of a nucleic acid polymerase for amplification, synthesisis, sequencing or copying. In the case of a double-stranded DNA molecule, denaturation of its strands to form a first and second strand is generally performed before amplification, synthesis or sequencing. A primer complementary to a portion of the template is hybridized to the template under appropriate conditions, and a polypeptide as described herein synthesizes a DNA molecule complementary to the template or portion thereof. Mismatch incorporation during the synthesis or extension of the newly synthesized DNA molecule may result in one or a number of mismatched base pairs. Thus, the synthesized DNA molecule need not be exactly complementary to the template. In the case of an RNA template, a DNA primer is hybridized to a strand of the template RNA and a polypeptide having reverse transcriptase activity is used to synthesize a complementary DNA.

Incorporating. The term “incorporating” refers to becoming part of a nucleic acid molecule or primer.

Amplification. As used herein “amplification” refers to any in vitro method for increasing the number of copies of a nucleotide sequence with the use of a DNA polymerase. Nucleic acid amplification results in the incorporation of nucleotides into a DNA molecule complementary to a template. The formed DNA molecule and its template can be used as templates to synthesize additional nucleic acid molecules. As used herein, one amplification reaction may consist of many rounds of DNA replication. DNA amplification reactions include, for example, PCR. One PCR reaction may consist of one or more e.g., 2, 3, 4, 5, 10, 15, 20, 25, 30, 50, 60, 70, 80, 90, 100 or more “cycles” of denaturation and synthesis of a DNA molecule.

Oligonucleotide. “Oligonucleotide” refers to a synthetic or natural molecule comprising a covalently linked series of nucleotides or nucleotide analogs. Such nucleotides or nucleotide analogs may be joined by a phosphodiester bond between the 3′ position of the pentose and the 5′ position of the pentose of the adjacent nucleotide. Also encompassed are molecules in which one or more internucleotide phosphate groups has been replaced by a different type of group, such as a peptide bond, a phosphorothioate group or a methylene group. Oligonucleotides may be synthetically prepared using protocols well known in the art.

Nucleotide. As used herein “nucleotide” refers to a base-sugar-phosphate combination. Nucleotides are monomeric units of a nucleic acid molecule (DNA and RNA). The term nucleotide includes deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs such as ddATP, ddCTP, ddGTP, ddITP and ddTTP) and their derivatives. A nucleotide may be unlabeled or detectable labeled by well known techniques. Detectable labels include, for example, radioactive isotopes, fluorescent labels, chemiluminescent labels, bioluminescent labels and enzyme labels. Nucleotides may also comprise one or more reactive functional groups. Labels may be attached to the functional group before, during and/or after use of the nucleotide in a nucleic acid synthesis, sequencing or amplification reaction.

A nucleotide may be unlabeled or detectably labeled by well known techniques. Detectable labels include, for example, radioactive isotopes, fluorescent labels, chemiluminescent labels and enzyme labels. Fluorescent labels of nucleotides include fluorescein, 5-carboxyfluorescein (FAM), 2′7′-dimethoxy-4′5-dichloro-6-carboxyfluorescein (JOE), rhodamine, 6-carboxyrhodamine (R6G), N,N, N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, and [dROX]ddTTP available from Perkin Elmer, Foster City, Calif.; FluoroLink DeoxyNucleotides, FluoroLink Cy3-dCTP, FluoroLink Cy5-dCTP, FluoroLink Fluor X-dCTP, Fluorolink Cy3-dUTP, and FluoroLink Cy5-dUTP available from Amersham, Arlington Heights, Ill.; Fluorescein-15-dATP, Fluorscein-12-dUTP, Tetramethyl-rhodamine-6-dUTP, IR₇₇₀-9-dATP, Fluorescein-12-ddUTP, Fluorescein-12-UTP, and Fluorescein-15-2′-dATP available from Boehringer Mannheim, Indianapolis, Ind.; and ChromaTide Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR -14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg.

Thermostable. As used herein “thermostable” refers to an activity of a molecule that is resistant to inactivation by heat. For example, DNA polymerases synthesize the formation of a DNA molecule complementary to a single-stranded DNA template by extending a primer in the 5′-to-3′ direction. This activity for mesophilic DNA polymerases may be inactivated by heat treatment. For example, T5 DNA polymerase activity is totally inactivated by exposing the enzyme to a temperature of 90° C. for 30 seconds. A thermostable activity is more resistant to heat inactivation than a corresponding mesophilic activity. Thermostable polymerases are relatively stable to heat and are capable of catalyzing the formation of DNA or RNA from a nucleic acid template. A thermostable DNA polymerase need not be totally resistant to heat inactivation, but exhibits reduced activity as a consequence of heat treatment. A thermostable DNA polymerase typically will also have a higher optimum temperature than common mesophilic DNA polymerases.

A polymerase is considered especially thermostable when it retains at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95% of its polymerase activity after heating, for example, at 95° C. for 30 minutes.

Fidelity. Fidelity refers to the accuracy of nucleic acid polymerization, or the ability of a nucleic acid polymerase to discriminate correct from incorrect substrates when synthesizing nucleic acid molecules complementary to a template. The higher the fidelity of a polymerase, the less the polymerase misincorporates nucleotides in the growing strand during nucleic acid synthesis. An increase or enhancement in fidelity results in a more faithful polymerase having decreased error rate (i.e., decreased misincorporation rate).

Hybridization. The terms “hybridization” and “hybridizing” refer to pairing of two complementary single-stranded portions of nucleic acid molecules (RNA and/or DNA) to a double stranded form. As used herein, two nucleic acid molecule portions may be hybridized, although the base pairing is not completely complementary. Accordingly, mismatched bases do not prevent hybridization of two nucleic acid molecule portions provided that appropriate hybridization and stringency conditions, well known in the art, are used.

The ability of two nucleotide sequences to hybridize to each other is based upon a degree of complementarity of the two nucleotide sequences, which is in turn based on the fraction of matched complementary nucleotide pairs. The more nucleotides in a given sequence that are complementary to another sequence, the greater the degree of hybridization of one to the other. The degree of hybridization also depends on the conditions of stringency which include temperature, solvent ratios, salt concentrations, and the like.

“Selective hybridization” pertains to conditions where the degree of hybridization of a polynucleotide to a target would require complete or nearly complete complementarity; a degree of complementarity sufficient to ensure that the polynucleotide binds specifically to the target relative to binding other nucleic acids present in the hybridization medium.

Stringent conditions. The phrase “stringent conditions” refers to conditions under which a nucleic acid will hybridize to a target sequence but will not hybridize or will hybridize to an insubstantial extent with a non-target sequence. Stringent conditions depend upon the length and sequence composition of the probe and target. Longer sequences and sequences with a higher G:C base content hybridize specifically at higher temperatures.

Generally, for a selected ionic strength of hybridization and wash buffer, stringent conditions include a temperature of about 5° C. below the calculated Tₘ for the specific probe and target sequences. Suitable hybridization and wash solutions are known to those skilled in the art and stringent conditions for a given probe and target pair can be determined without undue experimentation by adjusting the salt concentration and temperature until a single or small number of signals is obtained, for example, in a Southern blot. Stringent conditions are typically those that (1) employ low ionic strength and high temperature for washing, for example, 0.015 M NaCl/0.0015 M sodium citrate/0.1% sodium dodecyl sulfate (SDS) at 50° C., or (2) employ during hybridization a denaturing agent such as formamide, for example, 50% (vol/vol) formamide with 0.1% bovine serum albumin (BSA)/0.1% Ficoll/0.1% polyvinylpyrolidone/50 mM sodium phosphate buffer at pH 6.5 with 750 mM NaCl, 75 mM sodium citrate at 42° C. Another example is to use 50% formamide, 5×SSC (0.75 M NaCl and 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 42° C., with washes at 42° C. in 0.2×SSC and 0.1% SDS. Other suitable conditions include hybridization at 42° C. in a solution comprising 50% formamide, a first wash at 65° C. in 2×SSC and 1% SDS, and a second wash at 65° C. in 0.1×SSC; and hybridization in 6×SSC, 1% SDS, a first wash in 6×SSC, 1% SDS, and a final wash in a solution having a salt concentration of from about 0.05×SSC to about 0.3×SSC and about 0.05% SDS to about 1% SDS at a temperature of from about 50° C. to about 95° C.

3′-to-5′ Exonuclease Activity. “3′-to-5′exonuclease activity” is an enzymatic activity that results in the removal of the 3′-most nucleotide from a polynucleotide. This activity is often associated with DNA polymerases, and is thought to be involved in a DNA replication “editing” or correction mechanism in which incorrectly paired nucleotides are removed. Most DNA polymerases contain a 3′-5′ exonuclease activity in addition to polymerase activity. A T5 polymerase that lacks 3′-5′ exonuclease activity is disclosed in U.S. Pat. No. 5,270,179. Polymerases lacking this activity are particularly useful for, e.g., TA Cloning®.

A “DNA polymerase substantially reduced in 3′-5′exonuclease activity” is either (1) a mutated DNA polymerase that has about or less than 10%, or about or less than 1%, of the 3′-5′ exonuclease activity of the corresponding wild type enzyme, or (2) a DNA polymerase having a 3′-5′ exonuclease specific activity which is less than about 1 unit/mg protein, or preferably about or less than 0.1 units/mg protein. A unit of activity of 3′-5′ exonuclease is defined as the amount of activity that solubilizes 10 mmoles of substrate ends in 60 min at 37° C., assayed as described in the “BRL 1989 Catalogue & Reference Guide,” page 5, with HhaI fragments of lambda DNA 3′-end labeled with [³H]dTTP by terminal deoxynucleotidyl transferase (TdT). Protein is measured by the method of Bradford, Anal. Biochem. 72:248, 1976. As a means of comparison, natural, wild type T5-DNA polymerase (DNAP) or T5-DNAP encoded by pTTQ19-T5-2 (exo) (U.S. Pat. No. 5,270,179) has a specific activity of about 0.0001 units/mg protein, or 0.001% of the specific activity of the unmodified enzyme, a 10⁵-fold reduction.

5′-3′ Exonuclease Activity. “5′-3′exonuclease activity” is an enzymatic activity often associated with DNA polymerases such as E. coli DNA polI and polII. In many of the known polymerases, the 5′-3′ exonuclease activity is present in the N-terminal region of the polymerase (Ollis et al, Nature 313:762-766, 1985; Freemont et al., Proteins 1:66-73, 1986; Joyce, Curr. Opin. Struct. Biol. 1:123-129, 1991). Amino acid determinants of 5′-3′ exonuclease activity have been defined, e.g. for E. coli DNA polymerase I (Gutman et al., Nucl. Acids Res. 21:4406-4407, 1993). The 5′-exonuclease domain is dispensable for polymerase activity; e.g. as in the Klenow fragment of E. coli polymerase I. The Klenow fragment is a natural proteolytic fragment devoid of 5′-exonuclease activity (Joyce et al., J. Biol. Chem. 257:1958-1964, 1990). Polymerases lacking this activity are especially useful for DNA sequencing.

A DNA polymerase substantially reduced in 5′-3′ exonuclease activity is either (1) a mutated DNA polymerase that has about or less than 10%, or about or less than 1%, of the 5′-3′ exonuclease activity of the corresponding wild type enzyme, or (2) a DNA polymerase having a 5′-3′ exonuclease specific activity which is less than about 1 unit/mg protein, or preferably about or less than 0.1 units/mg protein.

Both 3′-5′ and 5′-3′ exonuclease activities can be observed on sequencing gels. Active 3′-5′ exonuclease activity will produce nonspecific ladders in a sequencing gel by removing nucleotides from the 5′-end of the growing primers. 5′-3′ exonuclease activity can be measured by following the degradation of radiolabeled primers in a sequencing gel. Thus, the relative amounts of these activities, e.g. by comparing wild type and mutant polymerases, can be determined with no more than routine experimentation.

Tzi DNA Polymerases and Variants Thereof

Native Tzi DNA polymerases (i.e., naturally occurring in Thermococcus zilligii) and variants thereof have a DNA-dependent DNA polymerase activity, and may also have one or more additional enzymatic activities, including an exonuclease activity (e.g., RNA-dependent RNA polymerase activity, 5′-3′ exonuclease activity and/or 3′-5′ exonuclease activity). Native and variant Tzi DNA polymerases may be purified and/or isolated from cells or organisms that express them. In some embodiments, native and variant Tzi DNA polymerases are substantially isolated from cells or organisms that express them. In other embodiments, native and variant Tzi DNA polymerases are substantially purified.

Native and variant Tzi DNA polymerases can be identified by homologous nucleotide and polypeptide sequence analyses using SEQ ID NO: 1 and 2, respectively. For example, performing a query on a database of nucleotide or polypeptide sequences can identify homologs of a known polypeptide. Homologous sequence analysis can involve BLAST or PSI-BLAST analysis of databases using known polypeptide amino acid sequences. Those proteins in the database that have greater than 35% sequence identity are candidates for further evaluation for suitability in the compositions and methods of the invention. If desired, manual inspection of such candidates can be carried out in order to narrow the number of candidates that can be further evaluated. Manual inspection is performed by selecting those candidates that appear to have domains conserved among known polypeptides.

A percent identity for any subject nucleic acid or amino acid sequence relative to another “target” nucleic acid or amino acid sequence can be determined as follows. First, a target nucleic acid or amino acid sequence can be compared and aligned to a subject nucleic acid or amino acid sequence, using the BLAST 2 Sequences (Bl2seq) program from the stand-alone version of BLASTZ containing BLASTN and BLASTP (e.g., version 2.0.14). The stand-alone version of BLASTZ can be obtained at <www.fr.com/blast> or at <www.ncbi.nlm.nih.gov>. Instructions explaining how to use BLASTZ, and specifically the Bl2seq program, can be found in the ‘readme’ file accompanying BLASTZ. The programs also are described in detail by Karlin et al. (1990) Proc. Natl. Acad. Sci. 87:2264; Karlin et al. (1993) Proc. Natl. Acad. Sci. 90:5873; and Altschul et al. (1997) Nucl. Acids Res. 25:3389.

Bl2seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per position cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acids (i.e., excluding gaps) from the target sequence that align with sequence from the subject sequence starting with any matched position and ending with any other matched position. A matched position is any position where an identical nucleotide or amino acid is present in both the target and subject sequence. Gaps of one or more positions can be inserted into a target or subject sequence to maximize sequence alignments between structurally conserved domains.

The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the Bl2seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90). In some embodiments, the amino acid sequence of a suitable homolog or variant has 40% sequence identity to the amino acid sequence of a known polypeptide. It will be appreciated that a nucleic acid or amino acid target sequence that aligns with a subject sequence can result in many different lengths with each length having its own percent identity. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

In some embodiments, the amino acid sequence of a homolog or variant has greater than 40% sequence identity (e.g., >80%, >70%, >60%, >50% or >40%) to the amino acid sequence of a known polypeptide.

The identification of conserved regions in a subject polypeptide can facilitate homologous polypeptide sequence analysis. Conserved regions can be identified by locating a region within the primary amino acid sequence of a subject polypeptide that is a repeated sequence, forms a secondary structure (e.g., alpha helices and beta sheets), establishes positively or negatively charged domains, or represents a protein motif or domain. See, e.g., the Pfam web site describing consensus sequences for a variety of protein motifs and domains at http://www.sanger.ac.uk/Pfam/ and http://genome.wustl.edu/Pfam/. A description of the information included at the Pfam database is described in Sonnhammer et al. (1998) Nucl. Acids Res. 26:320-322; Sonnhammer et al. (1997) Proteins 28:405-420; and Bateman et al. (1999) Nucl. Acids Res. 27:260-262. From the Pfam database, consensus sequences of protein motifs and domains can be aligned with the template polypeptide sequence to determine conserved region(s). Other methods for identifying conserved regions in a subject polypeptide are described, e.g., in Bouckaert et al. U.S. Ser. No. 60/121,700, filed Feb. 25, 1999.

Typically, polypeptides that exhibit at least about 35% amino acid sequence identity are useful to identify conserved regions. Conserved regions of related proteins sometimes exhibit at least 40% amino acid sequence identity (e.g., at least 50%, at least 60%, at least 70%, at least 80%, or at least 90% amino acid sequence identity). In some embodiments, a conserved region of target and template polypeptides exhibit at least 92, 94, 96, 98, or 99% amino acid sequence identity. Amino acid sequence identity can be deduced from amino acid or nucleotide sequence.

Variants include polypeptides which are at least 80%, 81%, 82%, 83%, 84%, 85%, 86%, 87%, 88%, 89%, 90%, 91%, 92%, 93%, 94%, 95%, 96%, 97%, 98%, or 99% identical to a the Tzi DNA polymerase of SEQ ID NO: 2, or to a conserved region thereof.

Some variants of native tzi DNA polymerases have an amino acid sequence with deletions, insertions, inversions, repeats and substitutions (e.g., conservative substitutions, non-conservative substitutions, type substitutions (for example, substituting one hydrophilic residue for another hydrophilic residue, but not a strongly hydrophilic for a strongly hydrophobic, as a rule), primary shifts, primary transpositions, secondary transpositions, and coordinated replacements) relative to a native tzi DNA polymerase (e.g., relative to SEQ ID NO:2). In some embodiments, the amino acid sequence of a variant corresponds to less than the full-length sequence (e.g. a conserved or functional domain) of a known polypeptide or homolog.

More than one amino acid (e.g., 2, 3, 4, 5, 6, 7, 8, 9, 10, etc.) can be deleted or inserted or can be substituted with another amino acid as described above (either conservative or nonconservative). Variants typically contain at least one amino acid substitution, deletion or insertion but not more than 50 (e.g., 15, 18, 20, 30, 35, 40, etc.) amino acid substitutions, deletions or insertions. In some embodiments, variants contain not more than 40, 30, or 20 amino acid substitutions, deletions or insertions. In additional embodiments, the variant contains at least one, but not more than 10, 9, 8, 7, 6, 5, 4, 3, 2 or 1 amino acid substitutions, deletions or insertions. In specific embodiments, the number of amino acid additions, substitutions and/or deletions in the polypeptide is 1-5, 5-25, 5-50, 10-50 or 50-150. In some embodiments, the amino acid substitutions are conservative substitutions.

Oligonucleotide directed mutagenesis can be used to create variant Tzi DNA polymerases. This technique allows for all possible base pair changes at any determined site along the encoding DNA molecule. In general, this technique involves annealing an oligonucleotide complementary (except for one or more desired mismatches) to a single stranded nucleotide sequence coding for the native DNA polymerase of interest. The mismatched oligonucleotide is then extended by DNA polymerase, generating a double stranded DNA molecule that contains the desired change in sequence on one strand. The changes in sequence can of course result in the deletion, substitution and/or insertion of an amino acid(s). The changed strand can be used as a template to form a double stranded polynucleotide. The double stranded polynucleotide can then be inserted into an appropriate expression vector, and a mutant polypeptide can thus be produced. The above-described oligonucleotide directed mutagenesis can be carried out using any technique known to those skilled in the art, for example, PCR. In one embodiment, mutations designed to alter the exonuclease activity do not adversely affect the polymerase activity.

One of skill in the art can make “conservatively modified variants” by making individual substitutions, deletions or additions to a polypeptide that alter, add or delete a single amino acid or a small percentage of amino acids in the encoded sequence where the alteration results in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see e.g., Creighton, Proteins (1984)).

Variant Tzi DNA polymerases include those in which the DNA polymerase and/or exonuclease activities of the enzyme are enhanced, reduced, substantially reduced or eliminated relative to the corresponding native Tzi DNA polymerase. Assays described herein and otherwise known in the art may routinely be applied to measure the ability of variants to exhibit an enzymatic activity, including the unit polymerase activity assay, rpsL fidelity assay and exo-nuclease assays described herein in Examples 4, 7 and 8, respectively.

Native and variant Tzi DNA polymerases may isolated/purified to have a DNA-dependent DNA polymerase specific activity of 1,000 to 100,000 units/mg protein. Thus, such polymerases may have a DNA-dependent DNA polymerase specific activity of, e.g., 2,000 to 50,000; 5,000 to 50,000; or 10,000 to 50,000 units/mg protein. One unit of DNA-directed DNA polymerase activity is the amount of enzyme required to incorporate 10 nmoles of dNTPs into acid insoluble product in 30 min (see Example 4).

Cloning and Expression of Native and Variant Tzi DNA Polymerases

Also provided are isolated nucleic acids encoding native Tzi DNA polymerases and variants thereof. To clone a gene encoding a native Tzi DNA polymerase, isolated DNA (e.g. cDNA) comprising the polymerase gene obtained from Tzi cells can be used to construct a recombinant DNA library in a vector. Prokaryotic vectors suitable for constructing such a plasmid library include plasmids such as those capable of replication in E. coli, including, but not limited to, pBR322, pET-26b(+), ColE1, pSC101, pUC vectors (pUC18, pUC19, etc., in Molecular Cloning, a Laboratory Manual, Cold Spring Harbor Laboratory Press, Cold Spring Harbor, N.Y., 1989). Bacillus plasmids include pC194, pC221, pC217, etc. (Glyczan, in Molecular Biology Bacilli, Academic Press, New York, pp 307-329. 1982). Suitable Streptomyces plasmids include pIJ101 (Kendall et al., J. Bacteriol. 169:4177-4183, 1987). Pseudomonas plasmids are reviewed by John et al. (Rad. Insec. Dis. 8:693-704, 1986) and Igaki (Jpn. J. Bacteriol. 33:729-742, 1978). Broad-host range plasmids or cosmids, such as pCP13 (Darzins et al., J. Bacteriol. 159:9-18, 1984) can also be used for the present invention.

Transformed E. coli cells can be plated and screened for the expression of a native or variant Tzi DNA polymerase by, e.g., transferring transformed cells to nitrocellulose membranes, lysing them, and then treating the membranes at 95° C. for 5 minutes to inactivate the endogenous E. coli enzyme. Different temperatures may be used to inactivate host polymerases depending on the host used and the temperature stability of the DNA polymerase desired to be cloned. DNA-directed DNA polymerase activity can then detected using well known techniques (e.g., Sanger et al., Gene 97:119-123, 1991).

Nucleic acids encoding native and variant Tzi DNA polymerases may be operably linked to a promoter and/or inserted into a vector (e.g., an expression vector). Such vectors be introduced and maintained in a prokaryotic host such as E. coli or other bacterium (e.g., Escherichia, Pseudomonas, Salmonella, Serratia, and Proteus). Eukaryotic hosts (e.g. insect, yeast, fungi and mammalian cells) also can be used for cloning and expressing native or variant Tzi DNA polymerases. The cloning and expression of native or variant Tzi DNA polymerases in prokaryotic and eukaryotic cells may be accomplished using well known tools and routine techniques.

Inducible or constitutive promoters are well known and may be used to optimize expression of native or variant Tzi DNA polymerases in a recombinant host. Similarly, high copy number vectors, well known in the art, may be used to achieve to enhance expression of native or variant Tzi DNA polymerases in a recombinant host. Native and variant Tzi DNA polymerases and polypeptides described herein can be produced by fermentation of a recombinant host expressing a cloned DNA polymerase. Native Tzi DNA polymerases also may be isolated from T. zilligii. Appropriate culture media and conditions can be selected according to the host strain used for expression and the composition of the culture medium. Antibiotics may also be added to the growth media to insure maintenance of nucleic acid vector encoding the DNA polymerase.

Host cells expressing native and variant Tzi DNA polymerases can be separated from liquid culture, for example, by centrifugation. In general, the collected cells are dispersed in a suitable buffer, and then broken down by ultrasonic treatment or by other well known procedures to allow extraction of the enzymes by the buffer solution. After removal of cell debris by ultracentrifugation or centrifugation, an expressed DNA polymerase can be isolated/purified by standard techniques (e.g., extraction, precipitation, chromatography, affinity chromatography, electrophoresis, etc.). Assays to monitor the presence of a DNA polymerase during isolation/purification are well known in the art.

Isolated Antibodies that Bind to Native and Variant Tzi DNA Polymerases

Native and variant Tzi DNA polymerases may be used to generate isolated antibodies, including polyclonal and monoclonal antibodies, using methods well known in the art. Such antibodies will bind specifically to the DNA polymerases, and may be useful for purification/isolation of native and variant Tzi DNA polymerases. Such antibodies also can be used for “Hot Start” nucleic acid amplification reactions, e.g., as described in U.S. Pat. No. 5,338,671.

Using Native and Variant Tzi DNA Polymerases

Native and variant Tzi DNA polymerases can be used for DNA sequencing, DNA labeling, DNA amplification and cDNA synthesis. Compositions and reactions for such nucleic acid synthesis, sequencing or amplification can include, in addition to a native or variant Tzi DNA polymerase, one or more dNTPs (dATP, dTTP, dGTP, dCTP), a nucleic acid template, an oligonucleotide primer, magnesium and buffer salts, and may also include other components (e.g., nonionic detergent). Sequencing compositions may also include one or more ddNTPs. The dNTPs or ddNTPs may be unlabeled or labeled with a fluorescent, chemiluminescent, bioluminescent, enzymatic or radioactive label.

Compositions comprising Tzi DNA polymerase may be formulated as described in copending U.S. application Ser. No. 09/741,664, the contents of which are incorporated herein in their entirety. Tzi DNA polymerase mutants devoid of or substantially reduced in 3′ to 5′ exonuclease activity and/or devoid of or substantially reduced in 5′ to 3′ exonuclease activity, may be useful for DNA sequencing, DNA labeling, and DNA amplification reactions and cDNA synthesis.

Thermostable native and variant Tzi DNA polymerases can be used for end-point PCR, qPCR (see e.g., U.S. Pat. Nos. 6,569,627; 5,994,056; 5,210,015; 5,487,972; 5,804,375; 5,994,076, the contents of which are incorporated by reference in their entirety), allele specific amplification, linear PCR, one step reverse transcriptase (RT)-PCR, two step RT-PCR, mutagenic PCR, multiplex PCR and the PCR methods described in co-pending U.S. patent application Ser. No. 09/599,594, the contents of which are incorporated by reference in their entirety.

Native and variant Tzi DNA polymerases can be used to prepare cDNA from mRNA templates (see e.g., U.S. Pat. Nos. 5,405,776 and 5,244,797, the disclosures of which are incorporated by reference in their entirety).

### EXAMPLES

### Example 1

### Cloning and Sequencing of a T. zilligii (Tzi) DNA Polymerase Gene

Sequences of Thermococcus DNA polymerases were retrieved from GenBank and aligned. Degenerate primers were designed to amplify a conserved region at the 3′ end of the polymerase genes to generate a 1.9 kb fragment. The amplified fragment was sequenced and determined to have significant identity to other Thermococcus DNA polymerases, thus confirming that the desired region was amplified. Additional degenerate primers were then designed to amplify 5′ sequences using the genomic walking PCR technique.

A native Tzi DNA polymerase gene was amplified using 4 degenerate primers and a short-range genomic walking library. A genomic walking library was prepared using 6 frequently cutting restriction enzymes (AluI, BsuRI, Bsp143I, RsaI, SmaI, Tsp509I). The chosen enzymes produced fragments with an average size of 120-1,000 bp. The primers used were:

| | |
|---|---|
| ARCHPOLF1: | |
| 5′-TACTACGGATACGCCAARGCNAGRTGGTA-3′ | (SEQ ID NO:3) |
| ARCHPOLF2: | |
| 5′-TACTACGGATACGCCAARGCNCGNTGGTA-3′ | (SEQ ID NO:4) |
| ARCHPOLR: | |
| 5′-GCGGGGAGAACCTGGTTNTCDATRTARTA-3′ | (SEQ ID NO:5) |
| THERMOPOLF1: | |
| 5′-TGGATTATGATCCTCGAYACNGAYTA-3′ | (SEQ ID NO:6) |
| THERMOPOLF2: | |
| 5′-AGGGAGTTCTTCCCNATGGARGC-3′ | (SEQ ID NO:7) |
| AN1.R1: | |
| 5′-GGCGGTAACGCTCTCGG-3′ | (SEQ ID NO:8) |
| AN1.R2: | |
| 5′-CCGGTGACACTATCCGCG-3′ | (SEQ ID NO:9) |
| AN1.R4: | |
| 5′-TAGAGCTTCCAGACCTCCACCG | (SEQ ID NO:10) |
| AN1.F1: | |
| 5′-GCGATACCCTTCGACGAGTTCG-3′ | (SEQ ID NO:11) |
| AN1.F3: | |
| 5′-AGATCCGAGACCATGCCCG-3′ | (SEQ ID NO:12) |

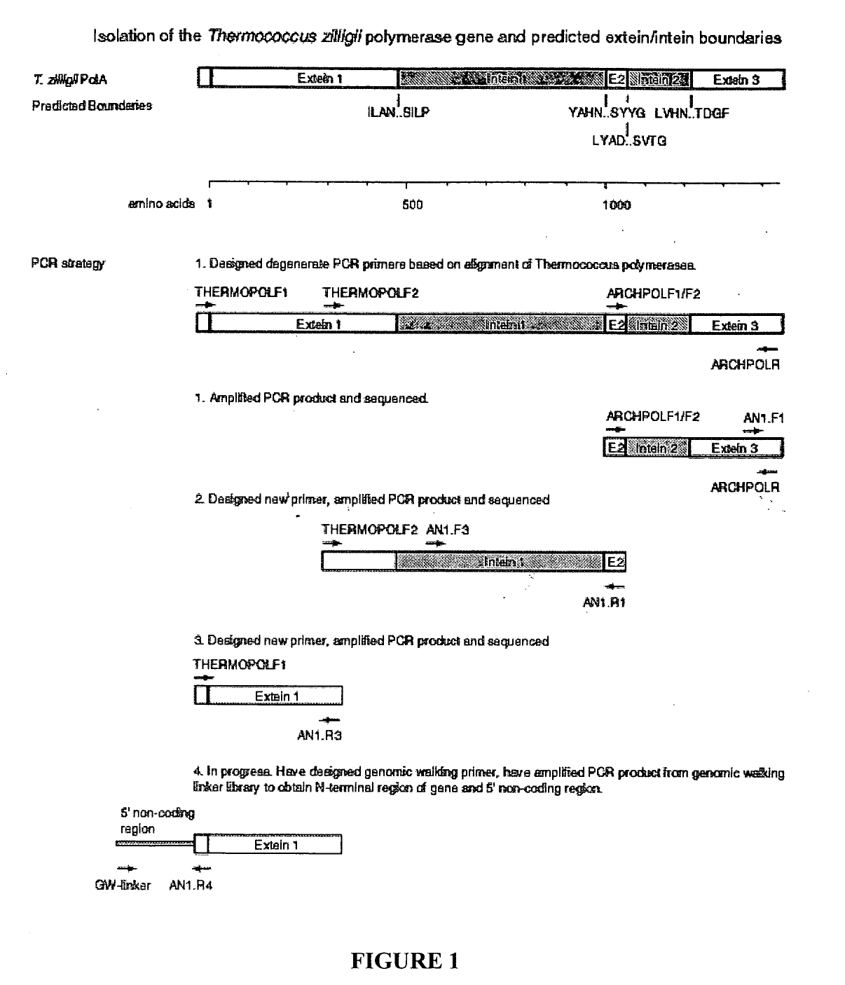

ARCHPOLF and R primers were used to amplify a 1160 bp fragment near the C terminus which included one intein. The genomic walking library was then used to amplify and sequence the C terminal portion of the gene. A degenerate primer (THERMOPOLF1) was then designed to bind to the start of the gene to be used with the AN1.R1 primer to amplify the 5′ portion. A final degenerate primer (THERMOPOLF2) was designed to bind to a site 1 kb downstream of the start of the gene. A 2.1 kb fragment was amplified using THERMOPOLF2 and AN1.R1. The beginning of the gene was amplified using the primer AN1.R4 which was designed based on the sequence of the 2.1 kb fragment. The Tzi DNA polymerase gene has three exteins and two inteins (the predicted extein/intein boundaries are shown in FIG. 1).

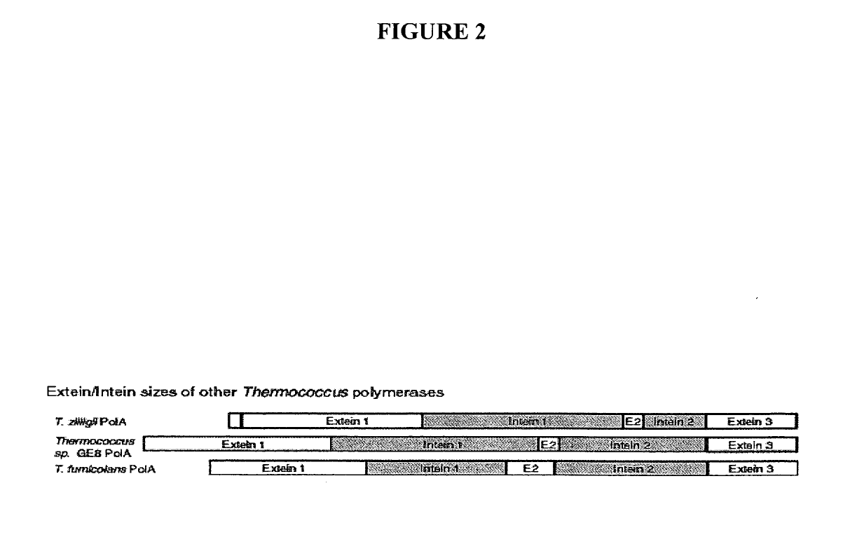

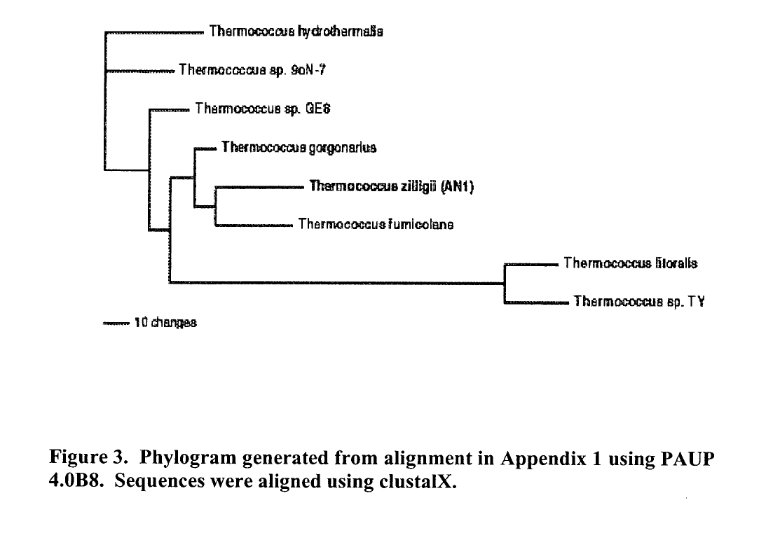

Comparison of the extein/intein sizes of Tzi DNA polymerase with those of other Thermococcus species is shown in FIG. 2. The deduced amino acid sequence of the Tzi DNA polymerase gene (SEQ ID NO: 2) is provided in Appendix 1, as is a comparison to the sequences of other thermococcus DNA polymerases (T. hydrothermali, SEQ ID NO:13; Thermococcus sp. 9oN-7, SEQ ID NO: 14; Thermococcus sp. GE8, SEQ ID NO: 15; T. fumicolans, SEQ ID NO: 16; T. gorgonarius, SEQ ID NO: 17; T. litoralis, SEQ ID NO: 18; Thermococcus sp. TY, SEQ ID NO: 19). Amino acid residues which differ from the consensus are shown in upper case font. Inteins are not shown in these sequences. The complete Tzi DNA polymerase gene sequence, including inteins, is provided in SEQ ID NO: 20, and the gene sequence without inteins is shown in SEQ ID NO: 1. The predicted Tzi DNA polymerase gene sequence (including inteins) is 4404 bp in length corresponding to 1467 amino acids with a molecular weight of 169.8 kDa. The phylogenetic relationships of the T. zilligii DNA polymerase to other Thermococcus DNA polymerases are shown in FIG. 3. This phylogram was generated from the sequence alignment in Appendix 1 using PAUP 4.0B8. Sequences were aligned using clustalX.

### Example 2

### Reconstruction and Cloning of a Native Tzi DNA Polymerase without Inteins

A strategy was devised to reconstruct an “intein-less” Tzi DNA polymerase. The primers used were:

AN1pETF:

(SEQ ID NO:21)

5′-GGGTGGGTCGACATGATCCTCGATGCTGAC-3′
AN1pETR:

(SEQ ID NO:22)

5′-CGGATTGCGGCCGCTCATGTCTTCGGTTTTAG-3′
Extein2F:

(SEQ ID NO:23)

5′-CCATCAAGATTCTGGCCAACAGTTATTACGGCTA-3′
Extein3F:

(SEQ ID NO:24)

5′-ACCGACGGTTTCTTTGC-3′
Extein1RB:

(SEQ ID NO:25)

5′-GGCCAGAATCTTGATGG-3′
Extein2RB:

(SEQ ID NO:26)

5′-GCAAAGAAACCGTCGGTATCCGCGTAAAGCACTT-3′

Primers Extein2F and Extein2RB have 17 bp overhangs complementary to the respective 5′ ends of Extein1R and Extein3F. Regeneration of double stranded DNA by PCR allows overlap extension between the 3′ends of PCR products encoding exteins 1 and 2, and exteins 2 and 3. Primers AN1pETF and AN1pETR incorporate the respective restriction sites SalI and NotI for directional in-frame ligation into plasmid pET26B (Novagen, No. 69862-3)

An intein-less Tzi DNA polymerase gene was reconstructed by the method outlined in FIG. 3, ligated into plasmid pET26B and transformed into E. coli. The E. coli transformants were grown at a temperature of 30° C. or less, and the plasmids/inserts were sequenced to confirm that the reconstructed gene was free of PCR-induced mutations. Intact plasmid DNA was then transformed into BL21-SI cells. Individual transformants were sequenced and confirmed to be free of PCR induced mutations. The length of this intein-less Tzi DNA polymerase gene was determined to be 2322 base pairs corresponding to 773 amino acids with a molecular weight of about 90 kDa, an isoelectric point of about 7.07 and a net charge of −2.

### Example 3

### Purification of Tzi DNA Polymerase

BL21CodonPlus host cells containing pET26B+Tzi pol were incubated at 37° C. in LB media supplemented with 25 μg/ml kanamycin, grown to an OD₆₀₀ of 1.0, and induced by isopropyl beta-D propyl thiogalactoside (IPTG) to a final concentration of 1 mM for three hours. Cells were harvested by centrifugation, resuspended in 3 ml of lysis buffer (50 mM Tris HCl, pH 7.5, 1 mM EDTA, 5 mM β-mercaptoethanol, 8% glycerol, 50 μg/ml Phenylmethylsulfonyl fluoride) per gram of wet cell paste and lysed by sonication (70-80% lysis based on OD₆₀₀). The lysate was heat-treated for 15 minutes at 65° C. then immediately placed on ice and sodium chloride (NaCl) was added to final concentration of 250 mM. Polyethylenimine (PEI; 2% v/v) was added dropwise to the lysate at 4° C. to final concentration of 0.15% (v/v) and mixed for 30 minutes at 4° C. The lysate was centrifuged for one hour using an SS-34 rotor at 17.5K rpm, and the supernatant was retained. Solid ammonium sulfate was added to the supernatant to ˜55% saturation while mixing at 4° C. The lysate was centrifuged for 30 minutes using an SS-34 rotor at 13K rpm, and the pellet was resuspended in low salt buffer (30 mM Tris HCl, pH 7.5, 1 mM EDTA, 1 mM DTT, 10% glycerol, 50 mM NaCl) and dialyzed against low salt buffer overnight.

The suspension was loaded onto a ten milliliter EMD-SO₃ column (1.6×5 cm) equilibrated with the low salt buffer. The column was washed with ten column volumes (cV) of low salt buffer and the protein was eluted with a 10 cV gradient from low salt buffer to 50% of high salt buffer (30 mM Tris HCl, pH 7.5, 1 mM EDTA, 1 mM DTT, 10% glycerol, 1000 mM NaCl), followed by a 3 cV wash at 50% high salt buffer. Four milliliter fractions were collected. Fractions were analyzed by SDS-PAGE (4-20% Novex Tris-glycine gel) stained with Novex SimplySafe stain according to manufacturer's manual. Fractions containing the desired protein band were further analyzed by the polymerase unit activity assay (described below). Appropriate fractions containing optimal activity were pooled and dialyzed against two liters of Resource Q low salt buffer (25 mM Tris-HCl (pH 8), 1 mM EDTA, 1 mM DTT, 10% glycerol, 50 mM NaCl).

The sample was loaded onto a one milliliter Resource Q column equilibrated with Resource Q low salt buffer. The column was washed with 10 cV of low salt buffer and eluted with 20 cV of linear gradient from low salt buffer to 25% of high salt buffer (25 mM Tris-HCl, pH 8, 1 mM EDTA, 1 mM DTT, 10% glycerol, 1000 mM NaCl, followed by an additional 20 cV wash at 25% of high salt buffer. One milliliter fractions were collected and analyzed by SDS-PAGE (4-20% Novex Tris-glycine gel) stained with Novex SimplySafe stain according to manufacturer's manual. Fractions containing the desired protein band were further analyzed by the polymerase unit activity assay. Appropriate fractions containing optimal activity were pooled and dialyzed against two liters of Storage buffer (20 mM Tris-HCl, pH 8, 40 mM KCl, 0.1 mM EDTA, 1 mM DTT, 50% glycerol, 0.5% NP-40, 0.5% Tween-20).

### Example 4

### Unit Assay for DNA Polymerase Activity

DNA polymerase activity was assessed by the standard incorporation rate of radiolabeled nucleotides into a nicked salmon testes DNA template. One polymerase unit corresponds to incorporation of 10 nmol of deoxynucleotides into acid-precipitable material in 30 min. at 74° C. under standard buffer conditions. The nucleotide incorporation into acid-insoluble fractions was measured by spotting an aliquot of the reaction onto a GF/C filter, washing the filter with trichloroacetic acid (TCA) solution, and counting the amount of radioactivity on the filter using a scintillation counter.

For a standard unit assay, 5 μl of a dilution of Tzi DNA polymerase was added to a set of 50 μl reactions. Each reaction contained 0.5 μg/μl of nicked salmon testes DNA and 0.2 mM of each dNTP (dATP, dCTP, dGTP, dTTP) in 1×Taq unit assay buffer (25 mM TAPS, pH 9.3, 50 mM KCl, 2 mM MgCl2, 1 mM DTT and 1 to 2 μCi [α-³²P] dCTP in a final volume of 50 μl per reaction. The reaction was initiated upon addition of the polymerase and transfer to a heating block equilibrated to 74° C. The reaction was continued for 10 min and terminated by adding 10 μl of 0.5 M EDTA to each of the 50 μl reactions on ice. 40 μl each of the mixtures was spotted onto a GF/C filter for TCA precipitation. Usually a dilution is needed so that the total amount of polymerase is below a saturation level. The saturation level could be empirically determined by using at least two dilutions of enzyme and correlating the unit activity at each dilution to the dilution factor. When both dilutions were below saturation, the activity should linearly correspond to the dilution factor.

TCA precipitation was performed as follows. The filters were washed in 10% TCA solution containing 1% sodium pyrophosphate for 15 min, in 5% TCA for 10 min three times, then in 95% ethanol for 10 min. The filters were dried under a heat lamp for 5 to 10 min and the radioactivity decay rate was measured in ScintiSafe Econo 1 scintillation cocktail (Fisher Scientific, part # SX20-5) using a Beckman scintillation counter (Model # LS 3801).

### Example 5

### Thermostability Determination

Polymerase thermostability was measured by incubating the enzyme diluted in the unit assay buffer without deoxynucleoside triphosphates or the template at a designated temperature. At various times during the incubation, an aliquot of the enzyme dilution was retrieved and stored until the assay. The remaining activities of the polymerase through the incubation were measured using the standard unit assay as described above. The time required for the unit activity to decrease to a half of the initial activity is called half life of the enzyme at the given temperature. There was almost no decrease in activity of this Tzi DNA polymerase during incubation at 95° C. for 60 min, suggesting that the half-life of this Tzi DNA polymerase at 95° C. would be several hours, making this enzyme one of the most thermostable known DNA polymerases.

### Example 6

### PCR Buffer Optimization

A Tris-SO₄ vs. Tris-HCl buffer comparison was performed in a pH range of 8.0 to 9.0 at 0.1 pH increments. In addition, a Tris-SO₄, Tris-HCl vs. Tris-acetate buffer evaluation was conducted with 0.2 pH increments between pH 8.0 and pH 9.0. A pH of 8.0 with 10 mM Tris-HCl appeared to be optimal.

For buffer optimization, titrations of MgCl₂, MgSO₄, and magnesium acetate were performed, with 1.2 mM MgSO₄ appearing optimal. KCl titrations also were performed, with 15 mM appearing optimal. (NH₄)₂SO₄ titrations also were performed, with 15 mM (NH₄)₂SO₄ appearing optimal.

Primer titrations were performed at 0.1, 0.2, 0.3, 0.4, 0.5, 0.6, 0.8, and 1.0 μM. Final concentrations of 0.3 and 0.4 μM appeared to increase yield, yet significant nonspecific binding appeared to take place at these concentrations. Thus, 0.2 μM primers was selected. Several dNTP titrations also were performed, with 0.2 mM appearing to be optimal.

Tzi unit titrations also were performed, with 2.5 U appearing optimal.

### Example 7

### PCR

Unless otherwise indicated, all the PCR reactions were performed following a standard protocol. PCR reactions were prepared in 50 μl reaction volumes containing 1× optimized Tzi buffer (10 mM Tris HCl, pH 8.0, 15 mM KCl, 15 mM (NH₄)₂SO₄, 1.2 mM MgSO₄), and 0.2 μM of each primer. The concentration of each of the four deoxynucleoside triphosphate (dNTPs) was 0.2 mM. Template concentration varied from 100 pg (for plasmids and cDNA) to 100 ng (genomic DNA) depending on the application. Two and one half units of Tzi DNA polymerase were used in a typical 50 μl reaction. Thermocycling was conducted using either the Perkin Elmer GeneAmp PCR System 9600 or the Perkin Elmer GeneAmp PCR System 2400. Standard PCR program: 94° C. for 2 minutes; 35 cycles of 94° C. for 15 seconds, then 50° C.-65° C. for 30 seconds (5 degrees below Tm), then 68° C. for 1 min/kb; and hold at 4° C.

Following the completion of thermocycling, PCR amplification products were mixed with 5 μl of 10× BlueJuice and aliquots (20%, or 10 μl, of total reaction volume per each lane) were analyzed by 0.8%-1.5% agarose gel electrophoresis with an ethidium bromide concentration of 0.5 μg/ml premixed in 0.5×TBE. The resulting gels were analyzed visually for specificity and yield among different samples.

### Example 8

### rpsL Fidelity Assay

Fidelity assays were performed based on streptomycin resistance exhibited by a rpsL mutation exhibits (Lackovich et al., 2001; Fujii et al., 1999). Briefly, pMOL 21 plasmid DNA (4 kb), containing the ampicillin resistance (Ap^{r}) and (rpsL) genes, was linearized with Sca I and standard PCR was performed on the linearized product using biotinylated primers annealed to the ends of the linearized template. Amplification was completed using 2.5 units of Tzi DNA polymerase for 25 cycles of amplification starting with 1 ng of the linearized template DNA. PCR cycling parameters were 94° C. for 2 min, followed by 25 cycles of 94° C. for 15 s, 58° C. for 30 s, and 68° C. for 5 min. PCR products were streptavidin-magnetic-bead-purified to isolate only the amplified product from the template.

Purified PCR products were analyzed on an agarose gel, and DNA concentration and template doubling was estimated based on the intensity of the band compared to standard bands with known amounts of DNA. The purified DNA was digested with MluI to cleave off the biotin label, ligated with T4 DNA ligase and transformed into MF101 competent cells. A portion of the transformants was plated on ampicillin plates to determine the total number of transformed cells and another portion was plated on ampicillin and streptomycin plates to determine the total number of rpsL mutants. Mutation frequency was determined by dividing the total number of mutations by the total number of transformed cells. The error rate was determined by dividing the mutation frequency by 130 (the number of amino acids that cause phenotypic changes for rpsL) and the template doubling. This fidelity assay showed that this Tzi DNA polymerase had 11 to 16 times higher fidelity than Taq DNA polymerase which was the same as that of KOD (Pfx) and Pfu Turbo DNA polymerase.

### Example 8

### Exo-Nuclease Activity

Like most archaeal DNA polymerases, this Tzi DNA polymerase has 3′-5′ exonuclase domain. The 3′ exonuclease activity is responsible for the proofreading activity of the polymerase and is therefore directly related to the fidelity of the enzyme. The 3′-5′ exonuclease activity of this Tzi DNA polymerase was tested by two different substrates, synthetic oligonucleotide with or without hairpin (the underlined sequence in KP_PALIN_{—}81 below indicates inverse repeat sequences that form the stem of the hairpin with its melting temperature was estimated to be 81° C.). KP_PALIN_cont lacks this hairpin structure. The hairpin structure was introduced to make the oligonucleotides preferable substrate for 5′-3′ exonuclease present in typeI polymerases, such as Taq DNA polymerase but not in archaeal polymerases.

KP_PALIN_81 (84 mer) (SEQ ID NO:27):
CTC CTG GAT CGA CTT CAG TCC GAC GAT GAT TAC ATC
AGC TCC TGG ATC GAC TTC ACT CCG CAC CCG CTA CCA
ACA ACA GTA CCC
KP_PALIN_cont (81 mer) (SEQ ID NO:28):
CTC CTG GAT CGA CTT CAG TCC GAT GAT TAG ATG TCG
TCC TGG ATC GAC TTC ACT CCG CAC CCG CTA CCA ACA
ACA GTA CCC

The oligonucleotide substrates were labeled with ³²P at the 5′ end using 10 units of T4 polynucleotide kinase and 10 μCi of [γ-³²P] ATP in 50 μl of 1×PNK exchange buffer. The reaction mix was incubated at 37° C. for 30 min and the reaction was terminated by incubating the mix at 70° C. for 10 min. Unincorporated nucleotides were removed by eluting the reaction mix through Amersham-Pharmacia Micro Spin G-25 column twice following the manufacturers instruction. About 100 pmol of the radio-labeled oligonucleotide was incubated with 60 units of polymerases in 120 μl of 1×PCR buffer (20 mM Tris-HCl, pH 8.4, 50 mM KCl) including 1.5 mM of MgCl₂ at 60° C. During incubation, 20 μl aliquots were taken out at 0, 2, 5, 10, and 30 min, and mixed with 10 μl of 3× formamide sequencing gel loading buffer and stored on ice. The samples were heated at 95° C. for 5 min and 10 μl of each was loaded onto an 15% polyacrylamide TBE urea gel and subjected to electrophoresis at 150 V for 45 min. The gel was dried and autoradiographed using Kodak BioMax MR X-ray film. Taq and Thermococcus kodakaraensis (KOD) polymerase (Novagen) were used in parallel with Tzi DNA polymerase as negative and positive control for 3′-5′ exonuclease activity. The results showed that this Tzi DNA polymerase had a 3′-5′ exonuclease activity as strong as, if not stronger than, that of KOD.

APPENDIX 1

| An alignment of Thermococcus DNA polymerases (without inteins). | | |
|---|---|---|
| Differences in sequence are shown in reverse text. | | |
| | 1 thermopolf1 80 | |
| T. hydrothermalis | .................................fepyiyallkddsaieevkkitaGrhgRVvKvKraekvkkkflgrpi | (SEQ ID NO:13) |
| Thermococcus sp. 9oN-7 | mildtdyiteNgkpvirvfkkengefkieydrTiepyFyallkddsaiedvkkvtaKrhgtVvKvKraekvQkkflgrpi | (SEQ ID NO:14) |
| Thermococcus sp. GE8 | mildtdyitedgkpvirvfkkengefkieydrNfepyFyallkddsaieevkkitaKrhgtVvKvKraekvkkkflgrpi | (SEQ ID NO:15) |
| T. fumicolans | mildtdyitedgRpvirvfkkengefkieydrdfepyiyallkddsaiedvkkitaSrhgtTvrvvraGkvkkkflgrpi | (SEQ ID NO:16) |
| T. zilligii (ANl) | .............pvirvfkkeKgefkidydrdfepyiyallkddsaiedikkitaerhgtTvrvTraeRvkkkflgrpv | (Amino acids 14-773 of SEQ ID NO:2) |
| T. Gorgonarius | mildtdyitedgkpvirifkkengefTidydrNfepyiyallkddsPiedvkkitaerhgtTvrvvraekvkkkflgrpi | (SEQ ID NO:17) |
| T. litoralis | mildtdyitKdgkpiirifkkengefkieLdPHfQpyiyallkddsaieeikAiKGerhgKTvrvLDaVkvRkkflgrEv | (SEQ ID NO:18) |
| Thermococcus sp. TY | mildtdyitkdgkpiirifkkengefkieLdPHfQpyiyallkddsaideikAiKGerhgKlvrvvDaVkvkkkflgrDv | (SEQ ID NO:19) |
| | 81 160 | |
| T. hydrothermalis | evwklyfthpqdvpairdEirRhSavvdiyeydipfakrylidkglipmegdeelkmmSfdietlyhegeefgTgpilmi | |
| Thermococcus sp. 9oN-7 | evwklyfNhpqdvpairdRirAhpavvidyeydipfakrylidkglipmegdeelTmlafdietlyhegeefgTgpilmi | |
| Thermococcus sp. GE8 | evwklyfthpqdvpairdkirehpavidiyeydipfakrylidkglipmegdeKlkmlafdietlyhegeefAegpilmi | |
| T. fumicolans | evwklyfthpqdvpairdkirehpavvdiyeydipfakrylidkglipmegdeelkmlafdietlyhegeefAegpilmi | |
| T. zilligii (ANl) | evwklyfthpqdvpairdkirehpavvdiyeydipfakrylidRglipmegdeelRmlafdietlyhegeefgegpilmi | |
| T. Gorgonarius | evwklyfthpqdvpairdkiKehpavvdiyeydipfakrylidkglipmegdeelkmlafdietlyhegeefAegpilmi | |
| T. litoralis | evwkllfEhpqdvpaMrGkirehpavvdiyeydipfakrylidkglipmegdeelkllafdietfyhegdefgKgEilmi | |
| Thermococcus sp. TY | evwkllfEhpqdvpaLrGkirehpavidiyeydipfakrylidkglipmegdeelklmafdietfyhegdefgKgEilmi | |
| | 161 240 | |
| T. hydrothermalis | syadeGEarvitwkKidlpyvevvstekemikrflkvvkekdpdvlityngdnfdfaylkkrCeklgikfTlRrdg..se | |
| Thermococcus sp. 9oN-7 | syadGSEarvitwkKidlpyvdvvstekemikrflRvvRekdpdvlityngdnfdfsylkkrCeElgikfTlgrdg..se | |
| Thermococcus sp. GE8 | syadeegarvitwkKvdlpyvdvvstekemikrflRvvkekdpdvlityngdnfdfaylkRrseklgvkfilgrdg..se | |
| T. fumicolans | syadeegarvitwkKidlpyvdvvstekemikrflkvvkekdpdvlityngdnfdfaylkkrseklgvkfilgrdg..se | |
| T. zilligii (ANl) | syadeegarvitwkNidlpyveSvstekemikrflkviQekdpdvlityngdnfdfaylkkrseTlgvkfilgrdg..se | |
| T. Gorgonarius | syadeegarvitwkNidlpyvdvvstekemikrflkvvkekdpdvlilyngdnfdfaylkkrseklgvkfilgreg..se | |
| T. litoralis | syadeeEarvitwkNidlpyvdvvsNeRemikrfVQvvkekdpdvlityngdnfdlPyllkrAeklgvRlvlgrdKehPe | |
| Thermococcus sp. TY | syadeeEarvitwkNidlpyvdvvsNeRemikrfVQivRekdpdvlityngdnfdlPyllkrAeklgvTlLlgrdKehPe | |
| | 241 320 | |
| T. hydrothermalis | pkiqrmgdrfavevkgrihfdlypvirrtinlptyleavyeavfgTpkekvyPeeiTTawetgeglervarysmedakv | |
| Thermococcus sp. 9oN-7 | pkiqrmgdrfavevkgrihfdlypvirrtinlptyleavyeavfgkpkekvyaeeiaqaweSgeglervarysmedakv | |
| Thermococcus sp. GE8 | pkiqrmgdrfavevkgrihfdlypvirrtinlptyleavyeaifgkpkekvyaeeiaTawetgeglervarysmedakv | |
| T. fumicolans | pkiqrmgdrfavevkgrihfdlypvirHtinlptyleavyeaifgQpkekvyaeeiaqawetgeglervarysmedakv | |
| T. zilligii (ANl) | pkiqrmgdrfavevkgrihfdlypvirrtinlpytleTvyeaifgQpkekvyaeeiaRaweSgeglervarysmedakA | |
| T. Gorgonarius | pkiqrmgdrfavevkgrihfdlypvirrtinlptyleavyeaifgQpkekvyaeeiaqawetgeglervarysmedakv | |
| T. litoralis | pkiqrmgdSfaveikgrihfdlfpvvrrtinlptyleavyeavlgkTkSkLGaeeiaAlwetEeSmKKLaQysmedaRA | |
| Thermococcus sp. TY | pkiHrmgdSfaveikgrihfdlfpvvrrtinlptyleavyeavlgkTkSkLGaeeiaAlwetEeSmKKLaQysmedaRA | |
| | 321 thermopolf2 400 | |
| T. hydrothermalis | tyelgReffpmeaqlsrligqslwdvsrsstgnlvewfllrkayernalapnkpderelarr.rgGyaggyvkeperglw | |
| Thermococcus sp. 9oN-7 | tyelgReffpmeaqlsrligqslwdvsrsstgnlvewfllrkayKrnelapnkpderelarr.rgGyaggyvkeperglw | |
| Thermococcus sp. GE8 | tfelgkeffpmeaqlsrligqslwdvsrsstgnlvewfllrkayernelapnkpderelarr.rQsyaggyvkeperglw | |
| T. fumicolans | tyelgReffpmeaqlsrlvgqsfwdvsrsstgnlvewyllrkayernelapnkpSGrelErr.rgGyaggyvkeperglw | |
| T. zilligii (ANl) | tyelgkeffpmeaqlsrlvgqslwdvsrsstgnlvewfllrkayernelapnkpderelarr.AEsyaggyvkepeKglw | |
| T. Gorgonarius | tyelgkeffpmeaqlsrlvgqslwdvsrsstgnlvewfllrkayernelapnkpderelarr.rEsyaggyvkeperglw | |
| T. litoralis | tyelgkeffpmeaEIAKligqsVwdvsrsstgnlvewyllrVayArnelapnkpdeEeYKrrlrTTyLggyvkepeKglw | |
| Thermococcus sp. TY | tyelgkeffpmeaEIAKligqsVwdvsrsstgnlvewyllrVayernelapnkpdeEaYRrrlrTTyLggyvkeperglw | |
| | 401 480 | |
| T. hydrothermalis | dnivyldfMslypsiiithnvspdtfnregckeydTapqvghkfckdVQgfipsllgAllderqkikkRmkaSidpLekk | |
| Thermococcus sp. 9oN-7 | dnivyldfrslypsiiithnvspdtlnregckeydvapEvghkfckdfpgfipsllgdlleerqkikRkmkatvdpLekk | |
| Thermococcus sp. GE8 | NnivyldfrslypsiiithnvspdtlnregckeydvapqvghkfckdfpgfipsllgdlleerqkikRkmRatidpvekk | |
| T. fumicolans | eniAyldfrslypsiiiShnvspdtlnregcGeydEapqvghRfckdfpgfipsllgdllderqkvkkHmkatvdpiekk | |
| T. zilligii (ANl) | enivyldyKslypsiiithnvspdtlnregcReydvapqvghRfckdfpgfipsllgdlleerqkvkkkmkatvdpieRk | |
| T. Gorgonarius | enivyldfrslypsiiithnvspdtlnregcEeydvapqvghkfckdfpgfipsllgdlleerqkvkkkmkatidpiekk | |
| T. litoralis | eniiyldfrslypsiivthnvspdtlEKegckNydvaplvgYRfckdfpgfipsllgdllAMrqDikkkmkStidpiekk | |
| Thermococcus sp. TY | eniAyldrfslypsiivthnvspdtlEregckNydvaplvgYkfckdfpgfipsllgellTMrqEikkkmkatidpiekk | |
| | 481 arechpolf1/f2 580 | |
| T. hydrothermalis | lldyrqKaikllansyygyygyaRarwyckecaesvtawgrDyiettiHeieeRfgfkvlyadtdgffatipgadaetvk | |
| Thermococcus sp. 9oN-7 | lldyrqraikilansfygyygyakarwyckecaesvtawgrEyieMVireLeekfgfkvlyadtdglHatipgadaetvk | |
| Thermococcus sp. GE8 | lldyrqraikilansyygyygyakarwycRecaesvtawgrSyiettireieekfgfkvlyadtdgffatipgadaetvk | |
| T. fumicolans | lldyrqraikilansfygyygyakarwyckecaesvtawgrQyiettMreieekfgfkvlyadtdgffatipgadaetvk | |
| T. zilligii (ANl) | lldyrqraikilansyygyygyaNarwycRecaesvtawgrQyiettMreieekfgfkvlyadtdgffatipgadaetvk | |
| T. Gorgonarius | lldyrqraikilansfygyygyTkarwyykecaesvtGwgrEyiettireieekfgfkvlyadtdgffatipgadaetvk | |
| T. litoralis | mldyrqraikLlansyygyMgyPkarwySkecaesvtawgrHyieMtireieekfgfkvlyadldgfyatipgEKPeLik | |
| Thermococcus sp. TY | mldyrqravkLlansyygyMgyPkarwySkecaesvtawgrHyieMtiKeieekfgfkvlyadtdgfyatipgEKPetik | |
| | 561 640 | |
| T. hydrothermalis | kkakeflkyinAklpglleleyegfyvrgffvtkkkyavideegkittrgleivrrdwseiaketqarvleailRhgdve | |
| Thermococcus sp. 9oN-7 | kkakeflkyinpklpglleleyegfyvrgffvtkkkyavideegkittrgleivrrdwseiaketqarvleailkhgdve | |
| Thermococcus sp. GE8 | kkaMeflkyinAklpglleleyegfyvrgffvtkkkyavideegkittrgleivrrdwseiaketqarvleailkhgdve | |
| T. fumicolans | kkaReflNyinpklpglleleyegfyRrgffvtkkkyavideegkittrgleivrrdwsevaketqarvleailRhgdve | |
| T. zilligii (ANl) | kkakeflNyinpRlpglleleyegfyRrgffvtkkkyavideeDkittrgleivrrdwseiaketqarvleailkhgdve | |
| T. Gorgonarius | kkakeflDyinAklpglleleyegfyKrgffvtkkkyavideeDkittrgleivrrdwseiaketqarvleailkhgdve | |
| T. litoralis | kkakeflNylnSklpglleleyegfyLrgffvtkkRyavideegRittrglevvrrdwseiaketqaKvleailkEgSve | |
| Thermococcus sp. TY | kkakeflkyinSklpglleleyegfyLrgffvAkkRyavideegRittrglevvrrdwseiaketqaKvleailkEDSve | |
| | 641 720 | |
| T. hydrothermalis | eavrivkdvteklskyevppeklviheqitrelkdykatgphvaiakrlaargikirpgtvisyivlkgsgrigdraipf | |
| Thermococcus sp. 9oN-7 | eavrivkevteklskyevppeklviheqitrdlRdykatgphvavakrlaargvkirpgtvisyivlkgsgrigdraipA | |
| Thermococcus sp. GE8 | eavrivkevteklskyevppeklviheqitrdlkdykatgphvavakrlaargikirpgtvisyivlkgsgrigdraipf | |
| T. fumicolans | eavrivkevteklskyevppeklviheqitrelkdykatgphvaiakrlaargikvrpgtvisyivlkgsgrigdrTipf | |
| T. zilligii (ANl) | eavrivkevteklsRyevppeklviYeqitrdlRdyRatgphvavakrlaargikirpgtvisyivlkgPgrvgdraipf | |
| T. Gorgonarius | eavrivkevteklskyevppeklviYeqitrdlkdykatgphvavakrlaargikirpgtvisyivlkgsgrigdraipf | |
| T. litoralis | KavEvvRdvVeklAkyRvpLeklviheqitrdlkdykatgphvaiakrlaargikvKpgtiisyivlkgsgKiSdrViLl | |
| Thermococcus sp. TY | KavEivkdvVeElAkyQvpLeklviheqitKdlSeykalgphvaiakrlaaKgikvrpgtiisyivlRgsgKiSdrViLl | |
| | 721 archpolr 778 | |
| T. hydrothermalis | defdpTkhrydaeyyienqvlpaverilKafgyKkeelryqktRqvglgawlkLkgkk | |
| Thermococcus sp. 9oN-7 | defdpTkhrydaeyyienqvlpaverilKafgyrkedlryqktkqvglgawlkVkgkk | |
| Thermococcus sp. GE8 | defdpakhKydaeyyienqvlpaverilrafgyrkedlryqktkqvglgawlkVkgkk | |
| T. fumicolans | defdpTkhrydaeyyienqvlpaverilKafgyKkedlryqktRqvglgawlkMgkk. | |
| T. zilligii (ANl) | defdpakhrydaeyyienqvlpaverilrafgyrkedlryqktkqAglgawlkpkT.. | |
| T. Gorgonarius | defdpakhKydaeyyienqvlpaverilrafgyrkedlryqktRqvglgawlkpkT.. | |
| T. litoralis | TeydpRkhKydPdyyienqvlpavLrilEafgyrkedlryqSSkqTglDawlkR.... | |
| Thermococcus sp. TY | SeydpKkhKydPdyyienqvlpavLrilEafgyrkedlKyqSSkqvglDawlkK.... | |

>AN1 PolA Reconstruct gene sequence (no inteins)

| | |
|---|---|
| ATGATCCTCGATGCTGACTACATCACCGAAGACGGA | (SEQ ID NO: 1) |
| AAGCCCGTCATAAGGGTCTTCAAGAAGGAAAAGGGC | |
| GAGTTTAAGATAGACTACGACAGGGACTTTGAGCCC | |
| TACATCTACGCCCTCCTGAAGGACGATTCCGCCATT | |
| GAGGACATCAAGAAGATCACCGCCGAGAGGCACGGC | |
| ACCACCGTTAGAGTTACCCGGGCGGAGAGGGTGAAG | |
| AAGAAGTTCCTCGGCAGGCCGGTGGAGGTCTGGAAG | |
| CTCTACTTCACCCACCCCCAGGACGTTCCCGCGATC | |
| AGGGACAAAATCAGGGAGCATCCGGCGGTTGTTGAC | |
| ATCTACGAGTACGACATACCCTTCGCGAAGCGCTAC | |
| CTCATAGACAGGGGCTTAATCCCTATGGAGGGGGAC | |
| GAGGAGCTCAGGATGCTCGCCTTCGACATCGAGACG | |
| CTCTACCATGAGGGGGAGGAGTTTGGCGAGGGGCCT | |
| ATCCTGATGATAAGCTACGCCGATGAAGAGGGGGCG | |
| CGCGTTATCACCTGGAAGAATATCGACCTCCCCTAC | |
| GTGGAGAGCGTTTCTACTGAGAAAGAGATGATAAAG | |
| CGCTTCCTCAAGGTAATCCAGGAGAAGGATCCGGAT | |
| GTGCTCATAACCTACAACGGCGACAACTTCGACTTT | |
| GCTTACCTCAAGAAGCGCTCAGAAACGCTCGGCGTC | |
| AAGTTCATCCTCGGAAGGGACGGGAGCGAACCGAAA | |
| ATTCAGCGCATGGGCGACCGCTTTGCAGTGGAGGTG | |
| AAGGGGAGAATACACTTCGACCTCTACCCGGTTATA | |
| AGGAGGACTATTAACCTCCCCACCTACACCCTCGAG | |
| ACAGTCTACGAGGCGATTTTCGGGCAACCAAAGGAG | |
| AAGGTCTACGCGGAAGAGATAGCGCGGGCCTGGGAG | |
| AGCGGGGAAGGCTTGGAAAGGGTGGCCCGCTATTCC | |
| ATGGAGGACGCAAAGGCAACTTACGAACTCGGAAAA | |
| GAGTTCTTCCCGATGGAGGCCCAGCTCTCGCGCCTC | |
| GTGGGCCAGAGCCTCTGGGATGTATCGCGCTCGAGC | |
| ACAGGAAACTTAGTTGAGTGGTTTCTCCTGAGGAAG | |
| GCCTACGAGAGGAACGAGCTCGCGCCAAACAAGCCG | |
| GACGAGAGGGAGTTAGCAAGGAGAGCGGAGAGCTAC | |
| GCGGGTGGATATGTCAAAGAGCCCGAAAAGGGGCTG | |
| TGGGAGAACATAGTCTACCTCGATTACAAATCTCTC | |
| TACCCCTCGATAATCATCACCCACAACGTCTCCCCT | |
| GATACCCTCAACAGGGAGGGCTGTAGGGAGTACGAC | |
| GTGGCACCTCAGGTGGGACACCGCTTCTGCAAGGAC | |
| TTCCCGGGCTTTATCCCGAGCCTCCTCGGGGACCTT | |
| TTGGAGGAGAGGCAGAAGGTAAAGAAGAAAATGAAG | |
| GCCACGGTGGACCCGATAGAGAGGAAGCTCCTCGAC | |
| TACAGGCAACGCGCCATCAAGATTCTGGCCAACAGT | |
| TATTACGGCTACTACGGCTACGCAAATGCCCGCTGG | |
| TACTGCAGGGAGTGCGCCGAGAGCGTTACCGCCTGG | |
| GGCAGGCAGTATATTGAAACCACGATGAGGGAAATA | |
| GAGGAGAAATTTGGCTTTAAAGTGCTTTACGCGGAT | |
| ACCGACGGTTTCTTTGCCACGATTCCCGGAGCGGAC | |
| GCCGAAACGGTCAAAAAGAAGGCTAAAGAATTCCTG | |
| AACTACATCAACCCCAGACTGCCCGGCCTGCTCGAG | |
| CTGGAGTACGAGGGCTTCTACAGGCGCGGCTTCTTY | |
| GTGACGAAGAAGAAGTACGCGGTTATAGACGAGGAG | |
| GACAAGATAACGACGCGCGGGCTGGAAATAGTAAGG | |
| CGCGACTGGAGCGAGATAGCGAAGGAGACGCAGGCG | |
| AGGGTTCTTGAGGCGATACTCAAGCACGGTGACGTC | |
| GAAGAGGCAGTAAGGATTGTCAAGGAGGTGACGGAA | |
| AAGCTGAGTAGGTACGAGGTTCCACCGGAGAAGCTC | |
| GTCATCTACGAGCAGATAACCCGCGACCTGAGGGAC | |
| TACAGGGCCACGGGGCCGCACGTGGCCGTTGCAAAA | |
| CGCCTCGCCGCGAGGGGGATAAAAATCCGGCCCGGG | |
| ACGGTCATAAGCTACATAGTGCTCAAAGGCCCGGGA | |
| AGGGTTGGGGACAGGGCGATACCCTTCGACGAGTTC | |
| GACCCTGCAAAGCACCGCTATGATGCGGAATACTAC | |
| ATCGAGAACCAGGTTCTTCCAGCGGTGGAGAGGATT | |
| CTGAGGGCCTTTGGTTACCGCAAAGAGGACTTGAGG | |
| TATCAGAAGACGAAGCAGGCCGGACTGGGGGCGTGG | |
| CTAAAACCGAAGACATGA | |

>AN1 PolA Reconstruct protein sequence
(no inteins)

| | |
|---|---|
| Met Ile Leu Asp Ala Asp Tyr Ile Thr | (SEQ ID NO: 2) |
| 1 5 10 15 | |
| Glu Asp Gly Lys Pro Val Ile Arg Val | |
| 20 25 30 | |
| Phe Lys Lys Glu Lys Gly Glu Phe Lys | |
| 35 40 45 | |
| Ile Asp Tyr Asp Arg Asp Phe Glu Pro | |
| 50 55 60 | |
| Tyr Ile Tyr Ala Leu Leu Lys Asp Asp | |
| 65 70 75 80 | |
| Ser Ala Ile Glu Asp Ile Lys Lys Ile | |
| 85 90 95 | |
| Thr Ala Glu Arg His Gly Thr Thr Val | |
| 100 105 110 | |
| Arg Val Thr Arg Ala Glu Arg Val Lys | |
| 115 120 125 | |
| Lys Lys Phe Leu Gly Arg Pro Val Glu | |
| 130 135 140 | |
| Val Trp Lys Leu Tyr Phe Thr His Pro | |
| 145 150 155 160 | |
| Gln Asp Val Pro Ala Ile Arg Asp Lys | |
| 165 170 175 | |
| Ile Arg Glu His Pro Ala Val Val Asp | |
| 180 185 190 | |
| Ile Tyr Glu Tyr Asp Ile Pro Phe Ala | |
| 195 200 205 | |
| Lys Arg Tyr Leu Ile Asp Arg Gly Leu | |
| 210 215 220 | |
| Ile Pro Met Glu Gly Asp Glu Glu Leu | |
| 225 230 235 240 | |
| Arg Met Leu Ala Phe Asp Ile Glu Thr | |
| 245 250 255 | |
| Leu Tyr His Glu Gly Glu Glu Phe Gly | |
| 260 265 270 | |
| Glu Gly Pro Ile Leu Met Ile Ser Tyr | |
| 275 280 285 | |
| Ala Asp Glu Glu Gly Ala Arg Val Ile | |
| 290 295 300 | |
| Thr Trp Lys Asn Ile Asp Leu Pro Tyr | |
| 305 310 315 320 | |
| Val Glu Ser Val Ser Thr Glu Lys Glu | |
| 325 330 335 | |
| Met Ile Lys Arg Phe Leu Lys Val Ile | |
| 340 345 350 | |
| Gln Glu Lys Asp Pro Asp Val Leu Ile | |
| 355 360 365 | |
| Thr Tyr Asn Gly Asp Asn Phe Asp Phe | |
| 370 375 380 | |
| Ala Tyr Leu Lys Lys Arg Ser Glu Thr | |
| 385 390 395 400 | |
| Leu Gly Val Lys Phe Ile Leu Gly Arg | |
| 405 410 415 | |
| Asp Gly Ser Glu Pro Lys Ile Gln Arg | |
| 420 425 430 | |
| Met Gly Asp Arg Phe Ala Val Glu Val | |
| 435 440 445 | |
| Lys Gly Arg Ile His Phe Asp Leu Tyr | |
| 450 455 460 | |
| Pro Val Ile Arg Arg Thr Ile Asn Leu | |
| 465 470 475 480 | |
| Pro Thr Tyr Thr Leu Glu Thr Val Tyr | |
| 485 490 495 | |
| Glu Ala Ile Phe Gly Gln Pro Lys Glu | |
| 500 505 510 | |
| Lys Val Tyr Ala Glu Glu Ile Ala Arg | |
| 515 520 525 | |
| Ala Trp Glu Ser Gly Glu Gly Leu Glu | |
| 530 535 540 | |
| Arg Val Ala Arg Tyr Ser Met Glu Asp | |
| 545 550 555 560 | |
| Ala Lys Ala Thr Tyr Glu Leu Gly Lys | |
| 565 570 575 | |
| Glu Phe Phe Pro Met Glu Ala Gln Leu | |
| 580 585 590 | |
| Ser Arg Leu Val Gly Gln Ser Leu Trp | |
| 595 600 605 | |
| Asp Val Ser Arg Ser Ser Thr Gly Asn | |
| 610 615 620 | |
| Leu Val Glu Trp Phe Leu Leu Arg Lys | |
| 625 630 635 640 | |
| Ala Tyr Glu Arg Asn Glu Leu Ala Pro | |
| 645 650 655 | |
| Asn Lys Pro Asp Glu Arg Glu Leu Ala | |
| 660 665 670 | |
| Arg Arg Ala Glu Ser Tyr Ala Gly Gly | |
| 675 680 685 | |
| Tyr Val Lys Glu Pro Glu Lys Gly Leu | |
| 690 695 700 | |
| Trp Glu Asn Ile Val Tyr Leu Asp Tyr | |
| 705 710 715 720 | |
| Lys Ser Leu Tyr Pro Ser Ile Ile Ile | |
| 725 730 735 | |
| Thr His Asn Val Ser Pro Asp Thr Leu | |
| 740 745 750 | |
| Asn Arg Glu Gly Cys Arg Glu Tyr Asp | |
| 755 760 765 | |
| Val Ala Pro Gln Val Gly His Arg Phe | |
| 770 | |
| Cys Lys Asp Phe Pro Gly Phe Ile Pro | |
| Ser Leu Leu Gly Asp Leu Leu Glu Glu | |
| Arg Gln Lys Val Lys Lys Lys Met Lys | |
| Ala Thr Val Asp Pro Ile Glu Arg Lys | |
| Leu Leu Asp Tyr Arg Gln Arg Ala Ile | |
| Lys Ile Leu Ala Asn Ser Tyr Tyr Gly | |
| Tyr Tyr Gly Tyr Ala Asn Ala Arg Trp | |
| Tyr Cys Arg Glu Cys Ala Glu Ser Val | |
| Thr Ala Trp Gly Arg Gln Tyr Ile Glu | |
| Thr Thr Met Arg Glu Ile Glu Glu Lys | |
| Phe Gly Phe Lys Val Leu Tyr Ala Asp | |
| Thr Asp Gly Phe Phe Ala Thr Ile Pro | |
| Gly Ala Asp Ala Glu Thr Val Lys Lys | |
| Lys Ala Lys Glu Phe Leu Asn Tyr Ile | |
| Asn Pro Arg Leu Pro Gly Leu Leu Glu | |
| Leu Glu Tyr Glu Gly Phe Tyr Arg Arg | |
| Gly Phe Phe Val Thr Lys Lys Lys Tyr | |
| Ala Val Ile Asp Glu Glu Asp Lys Ile | |
| Thr Thr Arg Gly Leu Glu Ile Val Arg | |
| Arg Asp Trp Ser Glu Ile Ala Lys Glu | |
| Thr Gln Ala Arg Val Leu Glu Ala Ile | |
| Leu Lys His Gly Asp Val Glu Glu Ala | |
| Val Arg Ile Val Lys Glu Val Thr Glu | |
| Lys Leu Ser Arg Tyr Glu Val Pro Pro | |
| Glu Lys Leu Val Ile Tyr Glu Gln Ile | |
| Thr Arg Asp Leu Arg Asp Tyr Arg Ala | |
| Thr Gly Pro His Val Ala Val Ala Lys | |
| Arg Leu Ala Ala Arg Gly Ile Lys Ile | |
| Arg Pro Gly Thr Val Ile Ser Tyr Ile | |
| Val Leu Lys Gly Pro Gly Arg Val Gly | |
| Asp Arg Ala Ile Pro Phe Asp Glu Phe | |
| Asp Pro Ala Lys His Arg Tyr Asp Ala | |
| Glu Tyr Tyr Ile Glu Asn Gln Val Leu | |
| Pro Ala Val Glu Arg Ile Leu Arg Ala | |
| Phe Gly Tyr Arg Lys Glu Asp Leu Arg | |
| Tyr Gln Lys Thr Lys Gln Ala Gly Leu | |
| Gly Ala Trp Leu Lys Pro Lys Thr | |

>AN1 PolA gene sequence (including inteins)

| | |
|---|---|
| ATGATCCTCGATGCTGACTACATCACCGAAGACG | (SEQ ID NO: 20) |
| GAAAGCCCGTCATAAGGGTCTTCAAGAAGGAAAA | |
| GGGCGAGTTTAAGATAGACTACGACAGGGACTTT | |
| GAGCCCTACATCTACGCCCTCCTGAAGGACGATT | |
| CCGCCATTGAGGACATCAAGAAGATCACCGCCGA | |
| GAGGCACGGCACCACCGTTAGAGTTACCCGGGCG | |
| GAGAGGGTGAAGAAGAAGTTCCTCGGCAGGCCGG | |
| TGGAGGTCTGGAAGCTCTACTTCACCCACCCCCA | |
| GGACGTTCCCGCGATCAGGGACAAAATCAGGGAG | |
| CATCCGGCGGTTGTTGACATCTACGAGTACGACA | |
| TACCCTTCGCGAAGCGCTACCTCATAGACAGGGG | |
| CTTAATCCCTATGGAGGGGGACGAGGAGCTCAGG | |
| ATGCTCGCCTTCGACATCGAGACGCTCTACCATG | |
| AGGGGGAGGAGTTTGGCGAGGGGCCTATCCTGAT | |
| GATAAGCTACGCCGATGAAGAGGGGGCGCGCGTT | |
| ATCACCTGGAAGAATATCGACCTCCCCTACGTGG | |
| AGAGCGTTTCTACTGAGAAAGAGATGATAAAGCG | |
| CTTCCTCAAGGTAATCCAGGAGAAGGATCCGGAT | |
| GTGCTCATAACCTACAACGGCGACAACTTCGACT | |
| TTGCTTACCTCAAGAAGCGCTCAGAAACGCTCGG | |
| CGTCAAGTTCATCCTCGGAAGGGACGGGAGCGAA | |
| CCGAAAATTCAGCGCATGGGCGACCGCTTTGCAG | |
| TGGAGGTGAAGGGGAGAATACACTTCGACCTCTA | |
| CCCGGTTATAAGGAGGACTATTAACCTCCCCACC | |
| TACACCCTCGAGACAGTCTACGAGGCGATTTTCG | |
| GGCAACCAAAGGAGAAGGTCTACGCGGAAGAGAT | |
| AGCGCGGGCCTGGGAGAGCGGGGAAGGCTTGGAA | |
| AGGGTGGCCCGCTATTCCATGGAGGACGCAAAGG | |
| CAACTTACGAACTCGGAAAAGAGTTCTTCCCGAT | |
| GGAGGCCCAGCTCTCGCGCCTCGTGGGCCAGAGC | |
| CTCTGGGATGTATCGCGCTCGAGCACAGGAAACT | |
| TAGTTGAGTGGTTTCTCCTGAGGAAGGCCTACGA | |
| GAGGAACGAGCTCGCGCCAAACAAGCCGGACGAG | |
| AGGGAGTTAGCAAGGAGAGCGGAGAGCTACGCGG | |
| GTGGATATGTCAAAGAGCCCGAAAAGGGGCTGTG | |
| GGAGAACATAGTCTACCTCGATTACAAATCTCTC | |
| TACCCCTCGATAATCATCACCCACAACGTCTCCC | |
| CTGATACCCTCAACAGGGAGGGCTGTAGGGAGTA | |
| CGACGTGGCACCTCAGGTGGGACACCGCTTCTGC | |
| AAGGACTTCCCGGGCTTTATCCCGAGCCTCCTCG | |
| GGGACCTTTTGGAGGAGAGGCAGAAGGTAAAGAA | |
| GAAAATGAAGGCCACGGTGGACCCGATAGAGAGG | |
| AAGCTCCTCGACTACAGGCAACGCGCCATCAAGA | |
| TTCTGGCCAACAGTATTCTGCCGGATGAGTGGAT | |
| CCCGCTACTCATTAATGGAAGGCTGAAACTGGTC | |
| AGAATCGGCGACTTTGTGGATAGTGCGATGAAAG | |
| AACTGAAGCCCATGAAAAGGGATGAAACGGAAGT | |
| CCTTGAAGTTTCTGGAATAGGTGCGATTTCCTTC | |
| AACAGGAAAACCAAGAGATCCGAGACCATGCCCG | |
| TCAGGGCCCTCCTGCGGCACCGCTACAGTGGAAA | |
| AGTGTACGGGATAAAGCTGTCCTCGGGGAGGAAG | |
| ATCAAAGTCACCGCGGGACACAGCCTCTTCACTT | |
| TCAGAGACGGGGAACTCGTGGAGATTAAGGGGGA | |
| GGAAATAAAACCCGGCGATTTCATAGCGGTTCCA | |
| GGAAGAATTAACCTCCCAGAAAGGCAGGAGAGGA | |
| TAAACCTCGTGGAGGTTCTCCTCGGCCTTCCTGA | |
| GGAGGAAACCGCCGACATCGTGCTGACGATCCCG | |
| GTTAAGGGACGTAGGAACTTCTTTAAAGGCATGC | |
| TGAGAACCCTTCGCTGGATTTTTGGGGAAGAGAA | |
| AAGGCCCGGGACGGCCAGGAGATACCTTGAACAC | |
| CTCCAAACGCTCGGCTACGTCAGGCTCGGGAAAA | |
| TCGGCTACGAAATAGTTAACGAGGAAGCCCTGAG | |
| GGACTACAGAGGGCTTTACGAGACTCTAACCGGA | |
| AAAGTGAAGTACAACGGCAATAAGAGGGAATACC | |
| TTGTGCACTTCAATGACCTGAGGGATATAATAAG | |
| ACTCATGCCAGAGAAGGAGCTTAAGGAATGGAAA | |
| GTTGGGACCCTCAACGGCTTCAGGATGGAGACTT | |
| CCATTGAAGTCAAGGAGGACTTTGCAAAGCTCCT | |
| CAGCTATTACGTCAGCGAGGGCTATGCAGGAAAG | |
| CAGAGAAGCCAGAAAAACGGGTGGAACTATTCAG | |
| TTAAGCTTTACAACAACGACCAAAACGTCCTTGA | |
| CGACATGGAAACGCTCGCCTCGAAGTTCTTCGGA | |
| AAGGTGAGACGCGGGAAGAATTACGTTGAGATCC | |
| CGAGGAAAATGGCCTACGTCCTCTTTGAGAGCCT | |
| TTGCGGTACTCTGGCCGAGAACAAACGGGTTCCT | |
| GAGATTATATTCACCTCCCCCGAGAGCGTGCGCT | |
| GGGCCTTCCTTGAGGGCTGCTTTATAGGGGACGG | |
| CGACCTTCATCCGGGCAAAGGGGTTAGACTTTCC | |
| ACGAAGAGCGAGGAACTGGTAAACGGTCTGGTCA | |
| TCTTACTCAACTCCCTTGGAGTTTCCGCCCTCAG | |
| GATATGGTTAGACAGCGGGGTTTACAGGGTTCTC | |
| GTCAACGAAGAGCTTCCGTTTTTAGACAAGGGCA | |
| AGAAAAAGACCCCCTACGTAACTTCAAAGGAAAT | |
| ACCGGAGGAGGCCTTTGGAAAACGGTTCCAGAGG | |
| AACATAAGCCTAGAAAAGCTCCGGGAGAAGGTTG | |
| AAAAGGGCGAGCCTGATGCGGAAAAGGTCAAGAG | |
| GGTCGTGTGGCTCCTTGAGGGAGATATAGTGCTT | |
| GATAGGGTTGAGGAAGTTGCAGTTGATGATTACG | |
| AGGGCTACGTCTACGACCTGAGCGTTGAAGAGAA | |
| CGAGAACTTCCTGGCAGGATTTGGAATGCTGTAC | |
| GCCCACAACAGTTATTACGGCTACTACGGCTACG | |
| CAAATGCCCGCTGGTACTGCAGGGAGTGCGCCGA | |
| GAGCGTTACCGCCTGGGGCAGGCAGTATATTGAA | |
| ACCACGATGAGGGAAATAGAGGAGAAATTTGGCT | |
| TTAAAGTGCTTTACGCGGATAGTGTCACCGGGGA | |
| CACCGAGGTAATCATCAGAAGGAACGGCAGGATC | |
| GAGTTCGTTCCAATCGAGAGACTCTTTGAGCACG | |
| TTGATTACCGGGTTGGTGAGAAAGAATACTGCGT | |
| TCTCAGCGGTGTTGAAGCACTGACACTCGACAAC | |
| AGGGGCAGGCTCGTTTGGAAGAAGGTTCCGTACG | |
| TCATGAGACATAAAACGGACAAGAGAATTTACCG | |
| CGTCTGGGTGACCAACAGCCGGTACCTGAACGTT | |
| ACGGAGGATCACTCGCTAATAGGTTATCTGGACG | |
| GAAAATACCTGGAGATAAGACCCGCTGATATCCC | |
| AAAAGATCCCGACATAAAGCTAATAACCCTCGCA | |
| TCCCCCGGGTTGCAGGAAGTCGCGCTCAAAACTC | |
| CCTCAAGGCTTGAAGAGATAACCTATGAGGGCTA | |
| CGTCTATGACATTGAAGTTGAAGGGRCCCACAGG | |
| TTCTTTGCCAACGGAATACTCGTTCACAACACCG | |
| ACGGTTTCTTTGCCACGATTCCCGGAGCGGACGC | |
| CGAAACGGTCAAAAAGAAGGCTAAAGAATTCCTG | |
| AACTACATCAACCCCAGACTGCCCGGCCTGCTCG | |
| AGCTGGAGTACGAGGGCTTCTACAGGCGCGGCTT | |
| CTTYGTGACGAAGAAGAAGTACGCGGTTATAGAC | |
| GAGGAGGACAAGATAACGACGCGCGGGCTGGAAA | |
| TAGTAAGGCGCGACTGGAGCGAGATAGCGAAGGA | |
| GACGCAGGCGAGGGTTCTTGAGGCGATACTCAAG | |
| CACGGTGACGTCGAAGAGGCAGTAAGGATTGTCA | |
| AGGAGGTGACGGAAAAGCTGAGTAGGTACGAGGT | |
| TCCACCGGAGAAGCTCGTCATCTACGAGCAGATA | |
| ACCCGCGACCTGAGGGACTACAGGGCCACGGGGC | |
| CGCACGTGGCCGTTGCAAAACGCCTCGCCGCGAG | |
| GGGGATAAAAATCCGGCCCGGGACGGTCATAAGC | |
| TACATAGTGCTCAAAGGCCCGGGAAGGGTTGGGG | |
| ACAGGGCGATACCCTTCGACGAGTTCGACCCTGC | |
| AAAGCACCGCTATGATGCGGAATACTACATCGAG | |
| AACCAGGTTCTTCCAGCGGTGGAGAGGATTCTGA | |
| GGGCCTTTGGTTACCGCAAAGAGGACTTGAGGTA | |
| TCAGAAGACGAAGCAGGCCGGACTGGGGGCGTGG | |
| CTAAAACCGAAGACATGA | |

What is claimed is:

1. An isolated native or variant Thermococcus zilligii (Tzi) DNA polymerase having an amino acid sequence at least 80% identical to SEQ ID NO: 2.

2. The isolated Tzi DNA polymerase of claim 1, having a molecular weight of about 90 kDa, and being stable at 95° C. for 60 minutes.

3. An expression vector encoding the Tzi DNA polymerase of claim 1.

4. A host cell comprising vector of claim 3.

5. An isolated monoclonal antibody that binds to the Tzi DNA polymerase of claim 1.

6. A method of synthesizing a double-stranded DNA molecule, comprising:
(a) hybridizing a primer to a first DNA molecule; and
(b) incubating said DNA molecule recited in (a) in the presence of one or more deoxy- and/or didexoyribonucleoside triphosphates and the Tzi DNA polymerase of claim 1 under conditions sufficient to synthesize a second DNA molecule complementary to all or a portion of said first DNA molecule.

7. A method of amplifying a double stranded DNA molecule, comprising:
(a) providing a first and second primer, wherein said first primer is complementary to a sequence at or near the 3′-terminus of the first strand of said DNA molecule and said second primer is complementary to a sequence at or near the 3′-terminus of the second strand of said DNA molecule;
(b) hybridizing said first primer to said first strand and said second primer to said second strand in the presence of the DNA polymerase of claim 1, under conditions such that the third strand complementary to said first strand and a fourth strand complementary to said second strand are synthesized;
(c) denaturing said first and third strands and said second and fourth strands; and
(d) repeating steps (a) to (c) one or more times.

8. A method of preparing cDNA from mRNA, comprising:
(a) contacting mRNA with an oligo(dT) primer or other complementary primer to form a hybrid; and
(b) contacting said hybrid formed in (a) with the DNA polymerase of claim 1 and dATP, dCTP, dGTP and dTTP, whereby a cDNA-RNA hybrid is obtained.

9. A method of preparing dsDNA from mRNA, comprising:
(a) contacting mRNA with an oligo(dT) primer or other complementary primer to form a hybrid; and
(b) contacting said hybrid formed in (a) with the DNA polymerase of claim 1, dATP, dCTP, dGTP and dTTP, and an oligonucleotide or primer which is complementary to the first strand cDNA; whereby dsDNA is obtained.
